(19) United States
(12) Patent Application Publication
(10) Pub. No.: US 2009/0181953 A1
(43) Pub. Date: Jul. 16, 2009

(54) COMPOUND FORMS AND USES THEREOF

(75) Inventors: Mahmoud Mirmehrabi, Laval (CA); Abdolsamad Tadayon, Montreal (CA); Subodh Deshmukh, White Plains, NY (US); Arianna Nencini, Castelnuovo Berardenga (IT); Riccardo Zanaletti, Colle Val d`Elsa (IT)

Correspondence Address: CHOATE, HALL & STEWART LLP, TWO INTERNATIONAL PLACE, BOSTON, MA, 02110, US

(73) Assignees: WYETH, Madison, NJ (US); SIENA BIOTECH S.P.A., Siena (IT)

(21) Appl. No.: 12/353,891

(22) Filed: Jan. 14, 2009

Related U.S. Application Data
(60) Provisional application No. 61/021,006, filed on Jan. 14, 2008.

(51) Int. Cl. A61K 31/551; C07D 403/12; A61P 25/00

(52) U.S. Cl. 514/218; 540/575

(57) ABSTRACT

The present invention provides, among other things, forms of a compound of formula 1. In some embodiments, the present invention provides salt forms and/or crystal forms. In some embodiments, the present invention provides solid forms. The present invention also provides methods of making and using provided forms.

### CROSS REFERENCE TO RELATED APPLICATIONS

This application claims priority to U.S. provisional patent application Ser. No. 61/021,006, filed Jan. 14, 2008, the entirety of which is hereby incorporated herein by reference.

### BACKGROUND

Modulators of α7 nicotinic acetylcholine receptors can have potent impact on neurological, psychiatric and/or inflammatory systems. In particular, certain receptor activators have been shown to be neuroprotective. Neuroprotective agents are useful in the treatment of a wide range of disorders.

### SUMMARY

The present invention provides new forms of a compound having the chemical structure presented in formula 1:

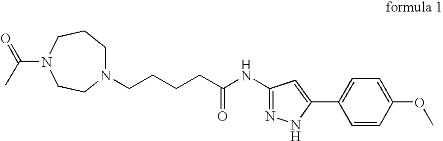

or pharmaceutically acceptable salts thereof. In some embodiments, the compound of formula 1 is in a salt form:

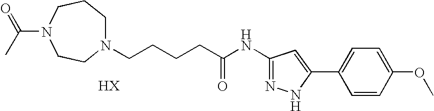

In some embodiments, the salt form is selected from the group consisting of acetate, citrate, D-glucoronate, fumarate, hydrochloride, oxalate, maleate, phosphate, salicylate, succinate, sulfate, tartarate forms. In some embodiments, the salt form is selected from the group consisting of fumarate, sulfate, D-glucoronate, and hydrochloride forms. In some embodiments, the salt form is the hydrochloride form:

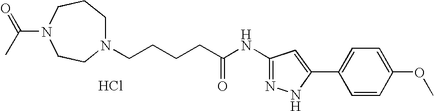

As will be appreciated by those of ordinary skill in the art, this compound includes an unsubstituted pyrazole ring; such rings are known to equilibrate in solution as mixtures of different tautomers as shown below:

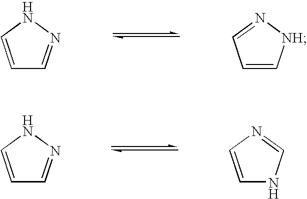

Provided forms of the compound may contain mixtures of tautomers or only a single tautomer.

The present invention encompasses the recognition that challenges may be encountered in preparing solid form of a compound of formula 1. The present invention provides solid forms of a compound of formula 1. The present invention also provides salt forms of a compound of formula 1. The present invention also provides crystal forms of certain compounds of formula 1.

The present invention also provides, among other things, compositions containing individual forms of a compound of formula 1, or mixtures thereof.

The present invention also provides methods of preparing a compound of formula I in a particular form or mixture of forms, and/or of preparing compositions containing such forms.

The present invention also provides methods of using particular forms of a compound of formula 1 and/or compositions containing them. In some embodiments, such forms and/or compositions are used to modulate α7 nicotinic acetylcholine receptors. In some embodiments, such forms and/or compositions are administered in vitro; in some embodiments, such forms are administered in vivo. In some embodiments, such forms and/or compositions are administered to a subject suffering from or susceptible to one or more neurological, psychiatric, and/or inflammatory disorders.

### DEFINITIONS

Hydrate: The term “hydrate”, as used herein, has its art-understood meaning, referring to a crystal form adopted by a particular compound in which either a stoichiometric or non-stoichiometric amount of water is incorporated into the crystal lattice.

In combination: The phrase “in combination”, as used herein, refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”.

Polymorph: As used herein, the term “polymorph” has its art-understood meaning, referring to one of a variety of different crystal structures that can be adopted by a particular compound.

Solvate: As used herein, the term “solvate” has its art-understood meaning, referring to a crystal form adopted by a particular compound in which either a stoichiometric or non-stoichiometric amount of solvent is incorporated into the crystal lattice.

Substantially free of: The term “substantially free of”, as used herein, means containing no more than an insignificant amount. In some embodiments, a composition or preparation is “substantially free of” a recited element if it contains less than 5%, 4%, 3%, 2%, or 1%, by weight of the element. In some embodiments, the composition or preparation contains less than 0.9%, 0.8%, 0.7%, 0.6%, 0.5%, 0.4%, 0.3%, 0.2%, 0.1% or less of the recited element. In some embodiments, the composition or preparation contains an undetectable amount of the recited element.

Therapeutically effective amount: As used herein, the term “therapeutically effective amount” means an amount that is expected or demonstrated to have a statistically significant likelihood of delaying onset of and/or reducing severity of one or more symptoms of a disease, disorder, or condition.

### DESCRIPTION OF THE DRAWING

FIG. 1 illustrates X-ray patterns observed for various salt forms.

FIGS. 2 and 3 present results of thermal studies performed on such salt forms.

FIGS. 4 and 5 present DVS data of sulfuric and phosphoric salts. The sulfuric salt form shows a low degree of hygroscopicity (about 0.4% water at 50% RH); the phosphoric salt form absorbed more water (about 2% water at 50% RH).

FIGS. 6-12 show characterization data for hydrochloride salts.

FIG. 13 illustrates the effect of pH and HCl equivalence on HCl salt formation.

FIG. 14 shows the effects of pH and HCl equivalence on HCl salt formation

FIGS. 15 and 16 depict conversion of higher HCl salts to mono-HCl forms.

FIG. 17 shows stability tests of a mixture of Crystal Forms I, II, and III and solvate I by solvent addition, resulting in Form I and the last melting point 200° C.

FIG. 18 shows a DSC scan of 5-(4-acetyl-1,4-diazepan-1-yl)-N-(5-(4-methoxyphenyl)-1H-pyrazol-3-yl)pentanamide hydrochloric salt Form I.

FIG. 19 shows a TGA thermogram of 5-(4-acetyl-1,4-diazepan-1-yl)-N-(5-(4-methoxyphenyl)-1H-pyrazol-3-yl)pentanamide hydrochloric salt Form I.

FIGS. 20a-b show X-ray diffraction pattern and data for 5-(4-acetyl-1,4-diazepan-1-yl)-N-(5-(4-methoxyphenyl)-1H-pyrazol-3-yl)pentanamide hydrochloric salt Form I.

FIG. 21 presents DVS isothermal analysis of 5-(4-acetyl-1,4-diazepan-1-yl)-N-(5-(4-methoxyphenyl)-1H-pyrazol-3-yl)pentanamide hydrochloric salt Form I.

FIG. 22 is a DSC scan of 5-(4-acetyl-1,4-diazepan-1-yl)-N-(5-(4-methoxyphenyl)-1H-pyrazol-3-yl)pentanamide hydrochloric salt Form II.

FIG. 23 is a TGA thermogram of 5-(4-acetyl-1,4-diazepan-1-yl)-N-(5-(4-methoxyphenyl)-1H-pyrazol-3-yl)pentanamide hydrochloric salt Form II.

FIGS. 24a-b show X-ray diffraction pattern and data for 5-(4-acetyl-1,4-diazepan-1-yl)-N-(5-(4-methoxyphenyl)-1H-pyrazol-3-yl)pentanamide hydrochloric salt Form II.

FIG. 25 presents a DVS isothermal analysis of 5-(4-acetyl-1,4-diazepan-1-yl)-N-(5-(4-methoxyphenyl)-1H-pyrazol-3-yl)pentanamide hydrochloric salt Form II.

### DESCRIPTION OF CERTAIN EMBODIMENTS

### Provided Forms of Compound of Formula 1

Compound of formula 1 has strong and specific activity as a modulator of α7 nicotinic acetylcholine receptors.

The present invention provides new forms of a compound of formula 1. In some embodiments, the present invention provides solid forms of a compound of formula 1. Indeed, the present invention encompasses the recognition that significant challenges can be encountered in preparing solid forms of a compound of formula 1. For example, the free base form and many salt forms of the compound do not readily adopt a solid state, but rather are typically liquid or semi-solid. Moreover, their behaviors may not be reproducible. The present invention encompasses the recognition that there is a need for new forms of a compound of formula 1, and also that there is a particular need for solid forms.

In some embodiments, the present invention provides salt forms of a compound of formula 1. For example, as described herein, the present invention provides acetic, citric, D-glucuronic, fumaric, hydrochloric, oxalic, maleic, phosphoric, salicylic, succinic, sulfuric, and tartaric acid forms. The present invention particularly provides solid forms of certain salts of a compound of formula 1. For example, the present invention provides solid forms of D-flucoronate, fumarate, hydrochloride, phosphate, and sulfate salts.

The present invention demonstrates that non-hygroscopic solid forms of a compound of formula 1 can be achieved. The present disclosure specifically exemplifies, for example, non-hygroscopic forms of hydrochloride, phosphate, and sulfate salt.

The present invention also demonstrates that discrete crystalline forms of certain salts of a compound of formula 1 can be achieved. Among others, the present invention specifically exemplifies a solvate form of a fumarate salt. The present invention further demonstrates that the hydrochloride salt of a compound of formula 1 can adopt a variety of different, distinguishable crystalline forms (e.g., Forms I-V). The present invention further demonstrates that certain such crystalline forms (e.g., Forms I and II) are hygroscopic.

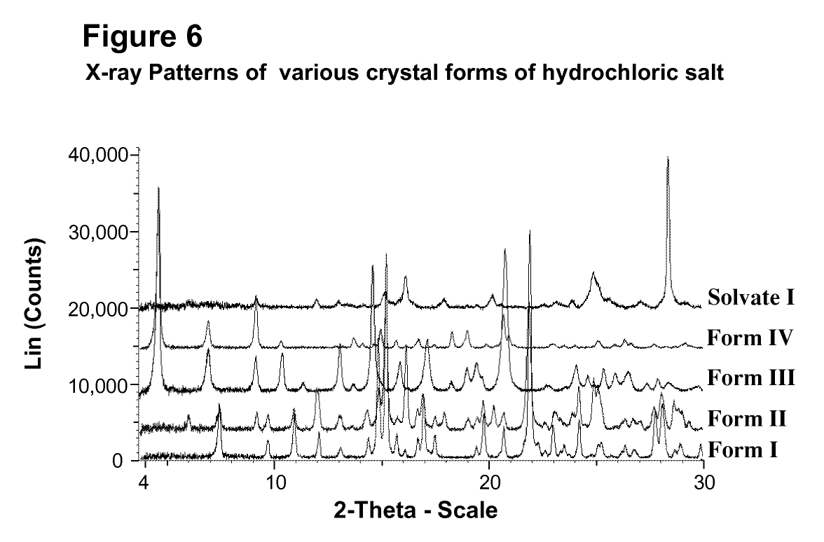

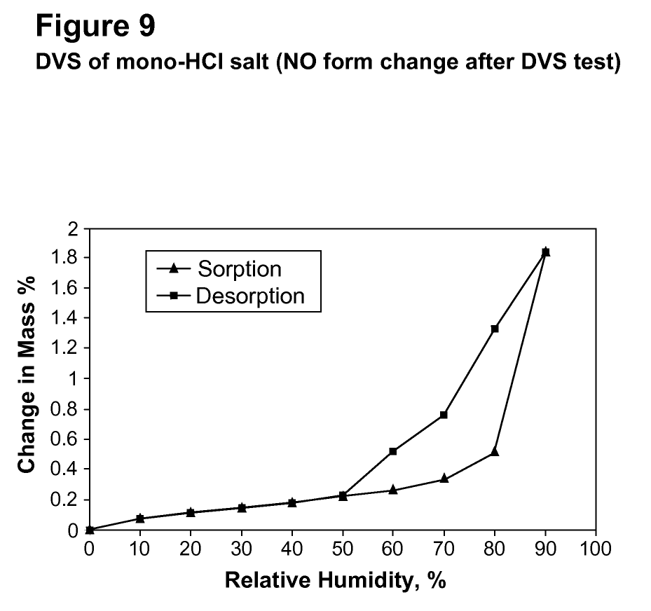

In some embodiments, the present invention provides Form I of the hydrochloride salt of a compound of formula 1. As described herein, Form I may be characterized by, for example, a melting point within the range of about 180-186° C. (e.g., about 185° C.), by a hygroscopicity profile as illustrated in FIG. 9 (e.g., gaining only about 0.5% moisture when equilibrated at RH less than or equal to 70%, and losing any moisture gained at higher RH without significant hysteresis upon decreasing RH), and/or by an X-ray diffraction pattern as shown for example in FIG. 6.

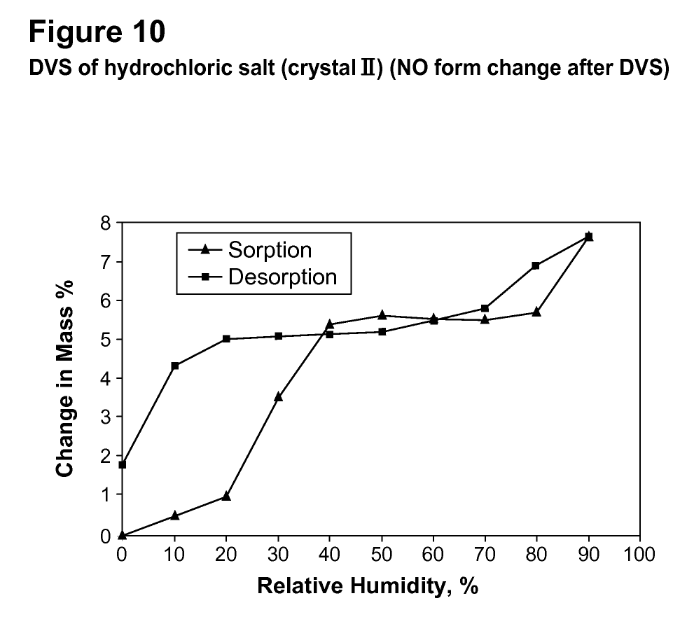

In some embodiments, the present invention provides Form II of the hydrochloride salt of a compound of formula 1. As described herein, Form II may be characterized by, for example, a melting point of about 167° C., by a hygroscopicity profile as illustrated in FIG. 10, and/or by an X-ray diffraction pattern as shown for example in FIG. 6.

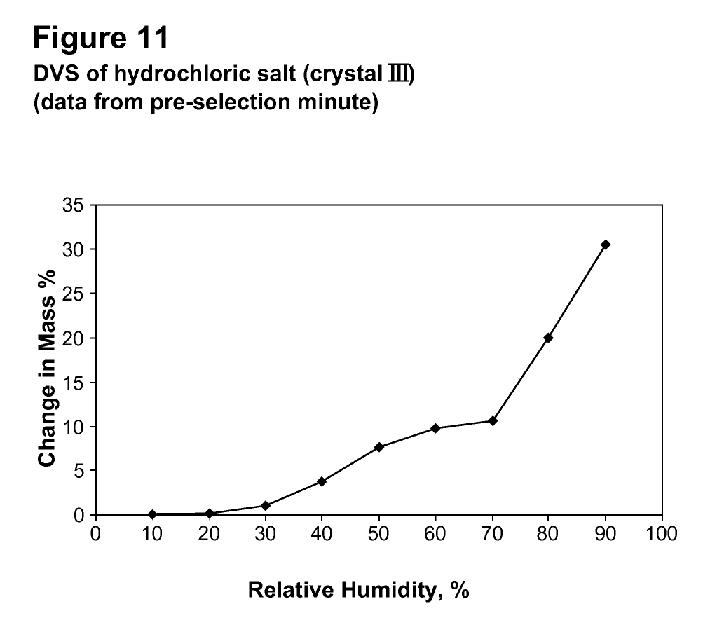

In some embodiments, the present invention provides Form III of the hydrochloride salt of a compound of formula 1. As described herein, Form III may be characterized by, for example, a melting point of about 119° C., by a hygroscopicity profile as illustrated in FIG. 11, and/or by an X-ray diffraction pattern as shown for example in FIG. 6.

In some embodiments, the present invention provides Form IV of the hydrochloride salt of a compound of formula 1. As described herein, Form IV may be characterized by, for example, a melting point of about 127° C., by a hygroscopicity profile as described herein, and/or by an X-ray diffraction pattern as shown for example in FIG. 6.

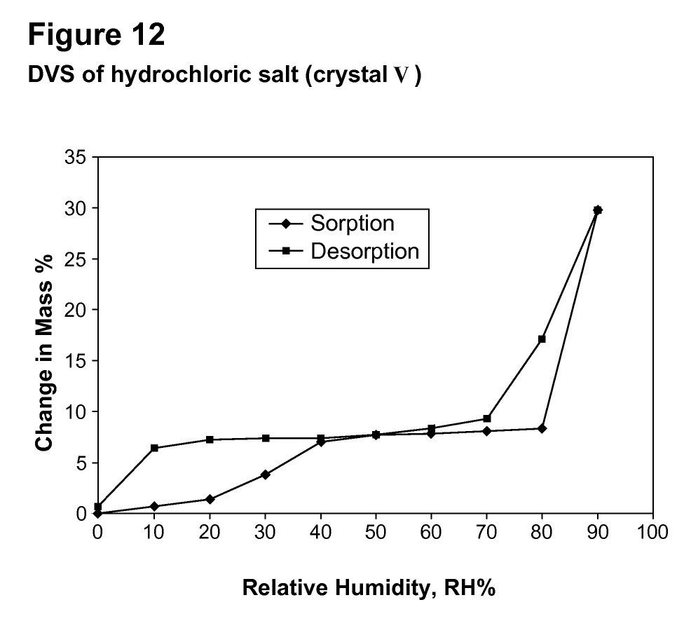

In some embodiments, the present invention provides a solvate form of the hydrochloride salt of a compound of formula 1. As described herein, this solvate form may be characterized by, for example, a series of endotherms, corresponding to 1) desolvation at about 100° C., 2) Form I at about 183° C., and 3) possibly another polymorph at about 200° C., by a hygroscopicity profile as illustrated in FIG. 12, and/or by an X-ray diffraction pattern as shown for example in FIG. 6.

Those of ordinary skill in the art will appreciate that X-ray diffraction patterns are often used to characterize individual crystal forms of a particular compound, and/or to detect the presence of the particular form in a complex composition. Those of ordinary skill in the art will further appreciate that precise identity of all peaks is not required to reveal a match of crystal form. Rather, presence or absence of particular characteristic peaks, and/or patterns of peaks and intensities, are typically both necessary and sufficient to characterize and/or identify a particular form.

X-ray peaks characteristic of a particular solid form (e.g., crystal form/polymorph) of the hydrochloride salt of a compound of formula 1 can readily be identified with reference to FIG. 6, which presents representative spectra for five different forms. For example, with reference to this FIG. 6, it can be seen that Form I may be characterized or identified, for example, based on the presence or absence of characteristic X-ray peaks at about 2θ of 15.3° and 21.9°, plus or minus about 0.3°, depending upon the machine and measurement method utilized; Form II may be characterized or identified, for example, based on the presence or absence of characteristic X-ray peaks at about 2θ of 20.2° and 24.9°, plus or minus about 0.3°, depending upon the machine and measurement method utilized. Those of ordinary skill, having referred to FIG. 6, will be able to identify alternative or additional X-ray peaks characteristic of one or more particular solid forms of the hydrochloride salt of this compound.

### Compositions Comprising Provided Forms of Compound of Formula 1

Any of the forms provided herein of a compound of formula 1 may be incorporated into a pharmaceutical composition. Typically, such a pharmaceutical composition will contain a therapeutically effective amount of the compound of formula 1, together with at least one pharmaceutically acceptable carrier or excipient.

In some embodiments, all of the compound of formula 1 that is present in a particular composition is present in a particular form; in some such embodiments, the composition is substantially free of any other form of the compound. In some embodiments, a composition comprises a compound of formula 1, present in a combination of different forms.

In some embodiments, a composition comprising a therapeutically effective amount of a compound of formula 1 is a solid composition; in some embodiments, such a solid composition is formulated for oral delivery.

Those of ordinary skill in the art will appreciate that pharmaceutical compositions as described herein may be prepared using any of a variety of standard materials, and any of a variety of known techniques as described, for example, in Remington's Pharmaceutical Sciences (e.g., Sixteenth Edition, E. W. Martin (Mack Publishing Co., Easton, Pa., 1980). Except insofar as a particular carrier or excipient (or other potential component of a pharmaceutical composition) is incompatible with the compound of formula 1 (in a form as described herein) and/or with other components of the composition, its use is contemplated to be within the scope of this invention.

To give but a few examples, materials that can typically serve as pharmaceutically acceptable carriers include but are not limited to ion exchanges, alumina, aluminum stearate, lecithin, serum proteins (e.g., human serum albumin), buffer substances (e.g., phosphates, glycine, sorbic acid, potassium sorbate, etc), partial glyceride mixtures of saturated vegetable fatty acids, water, salts or electrolytes (e.g., protamine sulfate, disodium hydrogen phosphate, potassium hydrogen phosphate, sodium chloride, zinc salts, colloidal silica, magnesium trisilicate, polyvinyl pyrrolidone, plyacrylate, waxes, polyethylene-polyoxypropylene-block polymers, wool fat, sugars (e.g., lactose, glucose, sucrose, etc), starches (e.g., corn starch, potato starch, etc.), cellulose and its derivatives (e.g., sodium carboxymethyl cellulose, ethyl cellulose, cellulose acetate, etc.), powdered tragacanth, malt, gelatin, talc, cocoa butter, suppository waxes, oils (e.g., peanut oil, cottonseed oil, safflower oil, sesame oil, olive oil, corn oil, soybean oil, etc.), glycols (e.g., propylene glycol, polyethylene glycol, etc.), esters (e.g., ethyl oleate, ethyl laurate, etc.), agar, buffering agents (e.g., magnesium hydroxide, aluminum hydroxide, etc.), alginic acid, pyrogen-free water, isotonic saline, Ringer's solution, ethyl alcohol, phosphate buffer solutions, lubricants (e.g., sodium laurel sulfate, magnesium stearate, etc.), coloring agents, releasing agents, coating agents, sweetening, flavoring agents, perfuming agents, preservatives, antioxidants, etc.

Pharmaceutical compositions as described herein may be formulated for delivery by any of a variety of routes including, for example, oral, nasal, rectal, parenteral, intracisternal, intravaginal, intraperitoneal, topical (e.g., by powder, oil, drops, etc.), bucal, etc. In many embodiments, the pharmaceutical compositions are solid compositions formulated, for example, for oral delivery.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound (and/or one or more metabolites thereof) into the bloodstream. The mode of administration may be left to the discretion of the practitioner.

In some embodiments, provided pharmaceutical compositions are administered orally; in some embodiments, provided pharmaceutical compositions are administered intravenously (e.g., after dissolution in an appropriate liquid carrier).

In some embodiments, it may be desirable to administer provided pharmaceutical compositions locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce a compound of formula 1 into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound of formula 1 can be formulated as a suppository, with traditional binders and excipients such as triglycerides.

In some embodiments, one or more compounds of formula 1 can be delivered in a vesicle, in particular a liposome (see Langer, Science 249:1527-1533, 1990 and Treat et al., Liposomes in the Therapy of Infectious Disease and Cancer 317-327 and 353-365, 1989).

In some embodiments, one or more compounds of formula 1 can be delivered in a controlled-release system or sustained-release system (see, e.g., Goodson, in Medical Applications of Controlled Release, vol. 2, pp. 115-138, 1984). Other controlled or sustained-release systems discussed in the review by Langer, Science 249:1527-1533, 1990 can be used. In some embodiments, a pump can be used (Langer, Science 249:1527-1533, 1990; Sefton, CRC Crit. Ref. Biomed. Eng. 14:201, 1987; Buchwald et al., Surgery 88:507, 1980; and Saudek et al., N. Engl. J. Med. 321:574, 1989). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61, 1983; Levy et al., Science 228:190, 1935; During et al., Ann. Neural. 25:351, 1989; and Howard et al., J. Neurosurg. 71:105, 1989).

As noted above, provided pharmaceutical compositions can optionally comprise a suitable amount of a physiologically acceptable excipient. Exemplary physiologically acceptable excipients can be liquids, such as water and oils, including those of petroleum, animal, vegetable, or synthetic origin, such as peanut oil, soybean oil, mineral oil, sesame oil and the like. For example, useful physiologically acceptable excipients can be saline, gum acacia, gelatin, starch paste, talc, keratin, colloidal silica, urea and the like. Alternatively or additionally, auxiliary, stabilizing, thickening, lubricating, and coloring agents can be used.

In some embodiments, a physiologically acceptable excipient that is sterile when administered to an animal is utilized. Such physiologically acceptable excipients are desirably stable under the conditions of manufacture and storage and will typically be preserved against the contaminating action of microorganisms. Water is a particularly useful excipient when a compound of formula 1 is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Provided pharmaceutical compositions, if desired, can also contain minor amounts of wetting or emulsifying agents, or pH buffering agents.

Liquid carriers may be used in preparing solutions, suspensions, emulsions, syrups, and elixirs. A compound of formula 1 can be dissolved or suspended in a pharmaceutically acceptable liquid carrier such as water, an organic solvent, a mixture of both, or pharmaceutically acceptable oils or fat. Such a liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Provided pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, pharmaceutical compositions in the form of a capsule are provided. Other examples of suitable physiologically acceptable excipients are described in Remington's Pharmaceutical Sciences 1447-1676 (Alfonso R. Gennaro, ed., 19th ed. 1995).

In some embodiments, a compound of formula 1 (in an appropriate form) is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the compound or pharmaceutically acceptable salt of the compound.

Capsules may contain mixtures of one or more compounds of formula 1 with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins.) Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, provided pharmaceutical compositions can be coated to delay disintegration and absorption in the gastrointestinal tract, thereby providing a sustained action over an extended period of time. Selectively permeable membranes surrounding an osmotically active driving compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In some embodiments, the excipients are of pharmaceutical grade.

In some embodiments, one or more compounds of formula 1 (in an appropriate form) can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where a compound of formula 1 is to be administered by infusion, it can be dispensed, for example, with an infusion bottle containing sterile pharmaceutical grade water or saline. Where a compound of formula 1 is administered by injection, an ampule of sterile water for injection or saline can be provided so that the ingredients can be mixed prior to administration.

In some embodiments, one or more compounds of formula 1 (in an appropriate form) can be administered transdermally through the use of a transdermal patch. Transdermal administrations include administrations across the surface of the body and the inner linings of the bodily passages including epithelial and mucosal tissues. Such administrations can be carried out using the present in lotions, creams, foams, patches, suspensions, solutions, and suppositories (e.g., rectal or vaginal).

Transdermal administration can be accomplished through the use of a transdermal patch containing one or more compounds of formula 1 (in an appropriate form) and a carrier that is inert to the compound or pharmaceutically acceptable salt of the compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as a semi-permeable membrane covering a reservoir containing a compound of formula 1 with or without a carrier, or a matrix containing the active ingredient.

One or more compounds of formula 1 (in an appropriate form) may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

One or more compounds of formula 1 (in an appropriate form) can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In some embodiments a controlled- or sustained-release composition comprises a minimal amount of a compound of formula 1 to treat or prevent one or more disorders, diseases or conditions associated with activity of α7 nicotinic acetylcholine receptors. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of one or more compounds of formula 1 that promptly produces a desired therapeutic or prophylactic effect, and gradually and continually release other amounts of the compound to maintain this level of therapeutic or prophylactic effect over an extended period of time. To maintain a constant level of the compound a body, the compound can be released from the dosage form at a rate that will replace the amount of the compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

In certain embodiments, provided pharmaceutical compositions deliver an amount of a compound of formula 1 that is effective in the treatment of one or more disorders, diseases, or conditions associated with activity (or inactivity) of α7 nicotinic acetylcholine receptors. According to the present invention, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. Effective dosage amounts described herein typically refer to total amounts administered; that is, if more than one compound of formula 1 is administered, the effective dosage amounts correspond to the total amount administered.

The effective amount of a compound of formula 1 for use as described herein will typically range from about 0.001 mg/kg to about 600 mg/kg of body weight per day, in some embodiments, from about 1 mg/kg to about 600 mg/kg body weight per day, in another embodiment, from about 10 mg/kg to about 400 mg/kg body weight per day, in another embodiment, from about 10 mg/kg to about 200 mg/kg of body weight per day, in another embodiment, from about 10 mg/kg to about 100 mg/kg of body weight per day, in another embodiment, from about 1 mg/kg to about 10 mg/kg body weight per day, in another embodiment, from about 0.001 mg/kg to about 100 mg/kg of body weight per day, in another embodiment, from about 0.001 mg/kg to about 10 mg/kg of body weight per day, and in another embodiment, from about 0.001 mg/kg to about 1 mg/kg of body weight per day.

In some embodiments, pharmaceutical compositions are provided in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. A unit dosage form can be, for example, a capsule or tablet itself, or it can be the appropriate number of any such compositions in package form. Such unit dosage form may contain, for example, from about 0.01 mg/kg to about 250 mg/kg, and may be given in a single dose or in two or more divided doses. Variations in the dosage will necessarily occur depending upon the species, weight and condition of the patient being treated and the patient's individual response to the medicament.

In some embodiments, the unit dosage form is about 0.01 to about 1000 mg. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg;

Precise amounts to be administered (whether in a particular unit dosage form or overall) may be adjusted, for example, by consideration of attributes of the subject (e.g., age, weight, lifestyle, etc), and/or of the disorder suffered by that subject (e.g., severity of disorder, etc.)

### Methods of Making Provided Forms of Compound of Formula 1 and Compositions Containing Them

Those of ordinary skill in the art will appreciate that provided forms of the compound of formula 1 may be prepared by any of a variety of available techniques. For example, in general, solid forms of a compound can be prepared according to Scheme 1 below:

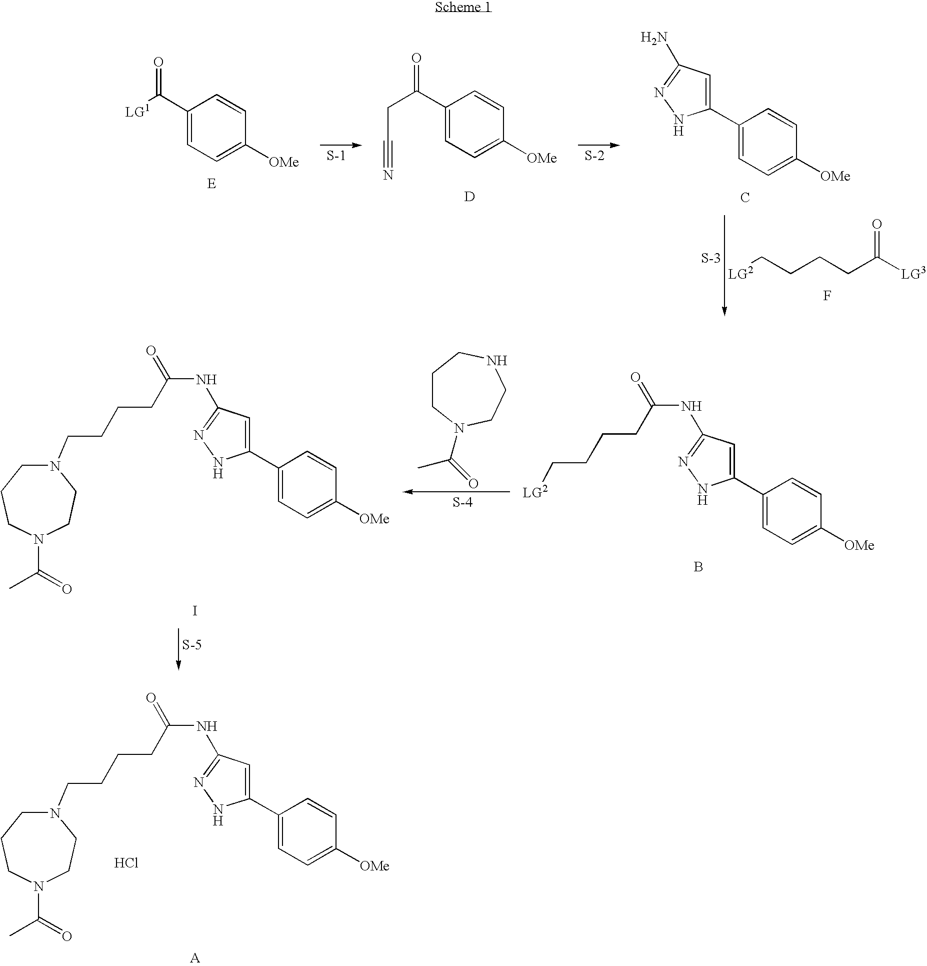

In some embodiments, Scheme 1 is used to prepare free base I. For example, in such embodiments, at step S-1, an aromatic ester of formula E is reacted, with a suitable base and acetonitrile to provide β-ketonitrile D. Examples of such suitable bases include NaH, LDA, NaHMDS, LHMDS, KHMDS, potassium t-amylate, BuLi, and NaOtBu. In certain embodiments, the base is LHMDS.

The LG¹ group of formula E is a suitable leaving group. A suitable leaving group is a chemical group that is readily displaced by a desired incoming chemical moiety. Suitable leaving groups are well known in the art, e.g., see, “Advanced Organic Chemistry,” Jerry March, 5^{th} Ed., pp. 351-357, John Wiley and Sons, N.Y. Such leaving groups include, but are not limited to, halogen, alkoxy, sulphonyloxy, optionally substituted alkylsulphonyl, optionally substituted alkenylsulfonyl, optionally substituted arylsulfonyl, and diazonium moieties. Examples of suitable leaving groups include chloro, iodo, bromo, fluoro, methanesulfonyl (mesyl), tosyl, triflate, nitro-phenylsulfonyl (nosyl), and bromo-phenylsulfonyl (brosyl). In some embodiments, LG¹ is OR, wherein R is C₁₋₆ alkyl. In certain embodiments, LG¹ is OR, wherein R is methyl.

At step S-2, β-ketonitrile D is reacted with hydrazine, to form aryl aminopyrazole C.

At step S-3, aryl aminopyrazole C is reacted with a compound of formula F to form a compound of formula B. The LG² group of formulae B and F is a suitable leaving group, as defined and described herein. In certain embodiments, the LG² group of formulae B and F is halogen, —OMs, —OTs, or —OTf.

The LG³ group of formula F is a suitable leaving group, as defined and described herein. In some embodiments, LG³ is —Cl.

In certain embodiments, the compound of formula F is selected from 5-bromovaleryl chloride or 5-iodovaleryl chloride. In some embodiments, the compound of formula F is 5-bromovaleryl chloride.

At step S-4, a compound of formula B is reacted in a suitable solvent with N-acetylhomopiperazine, optionally in the presence of a suitable base and/or iodine additive, to produce compound I, 5-(4-acetyl-1,4-diazepan-1-yl)-N-(5-(4-methoxyphenyl)-1H-pyrazol-3-yl)pentanamide. Suitable bases for step S-4 include pyridine, diisopropylethylamine, triethylamine, sodium bicarbonate, sodium carbonate, potassium carbonate, and combinations thereof. In certain embodiments, the base is potassium carbonate.

In certain embodiments, the iodide additive used in step S-4 is sodium iodide. In certain embodiments, the iodide source is potassium iodide.

At step S-5, compound I is reacted with hydrogen chloride, or an equivalent thereof, to form compound A, 5-(4-acetyl-1,4-diazepan-1-yl)-N-(5-(4-methoxyphenyl)-1H-pyrazol-3-yl)pentanamide hydrogen chloride. Suitable solvents for step S-5 include polar solvents such as C₁ to C₄ alcohols (e.g. ethanol, methanol, 2-propanol), water, acetone or combinations thereof. In certain embodiments, the solvent is selected from ethanol, water, acetone, or combination thereof. In some embodiments, the solvent a mixture of acetone, water, and ethanol.

Alternatively or additionally, various crystal forms of a particular compound (or salt form) of a compound of formula 1 can be prepared by recrystallization from a solvent system, under a particular set of conditions. In some embodiments, a particular crystal form may be prepared by seeding a solution of the compound with some existing crystal in the relevant form.

In some embodiments, a particular crystal or other solid form is isolated by removal of some or all of the solvent system, for example by one or more methods such as evaporation, distillation, filtration (e.g., nanofiltration, ultrafiltration, etc), reverse osmosis, absorption and/or reaction, by adding an anti-solvent such as heptane, by cooling, by drying (optionally under reduced pressure), or by combinations thereof.

### Use

### Therapeutic Regimens

Compounds of formula 1, and particularly compounds present in one or more of the forms described herein, as well as compositions containing them, are useful to modulate α7 nicotinic acetylcholine receptors. In some embodiments, such forms and/or compositions are administered in vitro; in some embodiments, such forms are administered in vivo. In some embodiments, such forms and/or compositions are administered to a subject suffering from or susceptible to one or more neurological, psychiatric, and/or inflammatory disorders.

That is, agents, such as compounds of formula 1, that bind to nicotinic acetylcholine receptors have been indicated as useful in the treatment and/or prophylaxis of various diseases and conditions, particularly psychotic diseases, neurodegenerative diseases involving a dysfunction of the cholinergic system, and/or conditions of memory and/or cognition impairment. Examples of such psychotic disorders include, for example, schizophrenia, anxiety, mania, depression, manic depression, etc; examples of neurodegenerative diseases include, for example, Tourette's syndrome, Parkinson's disease, Huntington's; examples of relevant cognitive disorders include, for example, Alzheimer's disease, Lewy Body Dementia, Amyotrophic Lateral Sclerosis, memory impairment, memory loss, cognition deficit, attention deficit, Attention Deficit Hyperactivity Disorder, etc.

Agents, such as compounds of formula 1, that bind to nicotinic acetylcholine receptors have also been shown to be useful, for example, in treatment of nicotine addiction, inducing smoking cessation, treating pain (i.e., analgesic use), providing neuroprotection, and treating jetlag. See, e.g., WO 97/30998; WO 99/03850; WO 00/42044; WO 01/36417; Holladay et al., J. Med. Chem., 40:26, 4169-94 (1997); Schmitt et al., Annual Reports Med. Chem., Chapter 5, 41-51 (2000); Stevens et al., Psychopharmatology, (1998) 136: 320-27; and Shytle et al., Molecular Psychiatry, (2002), 7, pp. 525-535.

Thus, in accordance with the invention, there is provided a method of treating a patient, especially a human, suffering from psychotic diseases, neurodegenerative diseases involving a dysfunction of the cholinergic system, and conditions of memory and/or cognition impairment, including, for example, schizophrenia, anxiety, mania, depression, manic depression, Tourette's syndrome, Parkinson's disease, Huntington's disease, and/or cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder) comprising administering to the patient an effective amount of a compound of formula 1 in a form as described herein.

Neurodegenerative disorders included within the methods provided herein include, but are not limited to, treatment and/or prophylaxis of Alzheimer's diseases, Pick's disease (Friedland, Dementia, (1993) 192-203; Procter, Dement Geriatr Cogn Disord. (1999) 80-4; Sparks, Arch Neurol. (1991) 796-9; Mizukami, Acta Neuropathol. (1989) 52-6; Hansen, Am J. Pathol. (1988) 507-18), diffuse Lewy Body disease, progressive supranuclear palsy (Steel-Richardson syndrome, see Whitehouse, J Neural Transm Suppl. (1987) 24:175-82; Whitehouse, Arch Neurol. (1988) 45(7):722-4; Whitehouse, Alzheimer Dis Assoc Disord. 1995; 9 Suppl 2:3-5; Warren, Brain. 2005 February; 128 (Pt 2):239-49), multisystem degeneration (Shy-Drager syndrome), motor neuron diseases including amyotrophic lateral sclerosis (Nakamizo, Biochem Biophys Res Commun. (2005) 330(4), 1285-9; Messi, FEBS Lett. (1997) 411(1):32-8; Mohammadi, Muscle Nerve. (2002) October; 26(4):539-45; Hanagasi, Brain Res Cogn Brain Res. (2002) 14(2):234-44; Crochemore, Neurochem Int. (2005) 46(5):357-68), degenerative ataxias, cortical basal degeneration, ALS-Parkinson's-Dementia complex of Guam, subacute sclerosing panencephalitis, Huntington's disease (Kanazawa, J Neurol Sci. (1985) 151-65; Manyam, J. Neurol. (1990) 281-4; Lange, J. Neurol. (1992) 103-4; Vetter, J. Neurochem. (2003) 1054-63; De Tommaso, Mov Disord. (2004) 1516-8; Smith, Hum Mol. Genet. (2006) 3119-31; Cubo, Neurology. (2006) 1268-71), Parkinson's disease, synucleinopathies, primary progressive aphasia, striatonigral degeneration, Machado-Joseph disease/spinocerebellar ataxia type 3, olivopontocerebellar degenerations, Gilles De La Tourette's disease, bulbar, pseudobulbar palsy, spinal muscular atrophy, spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, prion diseases (such as Creutzfeldt-Jakob, Gerstmann-Straussler-Scheinker disease, Kuru and fatal familial insomnia), and neurodegenerative disorders resulting from cerebral ischemia or infarction including embolic occlusion and thrombotic occlusion as well as intracranial hemorrhage of any type (including, but not limited to, epidural, subdural, subarachnoid and intracerebral), and intracranial and intravertebral lesions (including, but not limited to, contusion, penetration, shear, compression and laceration).

In addition, α7nACh receptor agonists, such as compounds of formula 1, can be used to treat age-related dementia and other dementias and conditions with memory loss including age-related memory loss, senility, vascular dementia, diffuse white matter disease (Binswanger's disease), dementia of endocrine or metabolic origin, dementia of head trauma and diffuse brain damage, dementia pugilistica, alcoholism related dementia (Korsakoff Syndrome) and frontal lobe dementia. See, e.g., WO 99/62505., Tomimoto Dement Geriatr Cogn Disord. (2005), 282-8; Tohgi—J Neural Transm. (1996), 1211-20; Casamenti, Neuroscience (1993) 465-71, Kopelman, Br J Psychiatry (1995) 154-73; Cochrane, Alcohol Alcohol. (2005) 151-4).

Thus, in accordance with the invention, there is provided a method of treating a patient, especially a human, suffering from age-related dementia and other dementias and conditions with memory loss comprising administering to the patient an effective amount of a compound of formula 1 in a form as described herein.

Thus, in accordance with a further embodiment, the present invention includes methods of treating patients suffering from memory impairment due to, for example, mild cognitive impairment due to aging, Alzheimer's disease, schizophrenia, Parkinson's disease, Huntington's disease, Pick's disease, Creutzfeldt-Jakob disease, depression, aging, head trauma, stroke, CNS hypoxia, cerebral senility, multiinfarct dementia and other neurological conditions, as well as HIV and cardiovascular diseases, comprising administering an effective amount of a compound of formula 1 in a form as described herein.

Amyloid precursor protein (APP) and Aβ peptides derived therefrom, e.g., AβM0, APi-42, and other fragments, are known to be involved in the pathology of Alzheimer's disease. The Aβi-42 peptides are not only implicated in neurotoxicity but also are known to inhibit cholinergic transmitter function. Further, it has been determined that Aβ peptides bind to α7nACh receptors. Thus, agents, such as compounds of formula 1, which block the binding of the Aβ peptides to α-7 nAChRs are useful for treating neurodegenerative diseases. See, e.g., WO 99/62505. In addition, stimulation α7nACh receptors can protect neurons against cytotoxicity associated with Aβ peptides. See, e.g., Kihara, T. et al., Ann. Neurol., 1997, 42, 159. Thus, in accordance with an embodiment of the invention there is provided a method of treating and/or preventing dementia in an Alzheimer's patient which comprises administering to the subject a therapeutically effective amount of a compound of formula 1, in a form as described herein, to inhibit the binding of an amyloid beta peptide (preferably, Aβ 1-42) with nACh receptors, preferable α7nACh receptors, most preferably, human α7nACh receptors (as well as a method for treating and/or preventing other clinical manifestations of Alzheimer's disease that include, but are not limited to, cognitive and language deficits, apraxias, depression, delusions and other neuropsychiatric symptoms and signs, and movement and gait abnormalities).

The present invention also provides methods for treating other amyloidosis diseases, for example, hereditary cerebral angiopathy, normeuropathic hereditary amyloid, Down's syndrome, macroglobulinemia, secondary familial Mediterranean fever, Muckle-Wells syndrome, multiple myeloma, pancreatic- and cardiac-related amyloidosis, chronic hemodialysis anthropathy, and Finnish and Iowa amyloidosis.

In addition, nicotinic receptors have been implicated as playing a role in the body's response to alcohol ingestion. Thus, agonists for α7nACh receptors can be used in the treatment of alcohol withdrawal and in anti-intoxication therapy. Thus, in accordance with an embodiment of the invention there is provided a method of treating a patient for alcohol withdrawal or treating a patient with anti-intoxication therapy comprising administering to the patient an effective amount of a compound of formula 1, in a form as described herein.

Agonists for the α7nACh receptor subtypes can also be used for neuroprotection against damage associated with strokes and ischemia and glutamate-induced excitotoxicity. Thus, in accordance with an embodiment of the invention there is provided a method of treating a patient to provide for neuroprotection against damage associated with strokes and ischemia and glutamate-induced excitotoxicity comprising administering to the patient an effective amount of a compound of formula 1 in a form as described herein.

Agonists for the α7nACh receptor subtypes can also be used in the treatment of nicotine addiction, inducing smoking cessation, treating pain, and treating jetlag, obesity, diabetes, sexual and fertility disorders (eg. Premature ejaculation or vaginal dryness, see U.S. Pat. No. 6,448,276), drug abuse (Solinas, Journal of Neuroscience (2007) 27(21), 5615-5620), and inflammation. Thus, in accordance with an embodiment of the invention there is provided a method of treating a patient suffering from nicotine addiction, pain, jetlag, obesity and/or diabetes, or a method of inducing smoking cessation in a patient comprising administering to the patient an effective amount of a compound of formula 1 in a form as described herein.

The inflammatory reflex is an autonomic nervous system response to an inflammatory signal. Upon sensing an inflammatory stimulus, the autonomic nervous system responds through the vagus nerve by releasing acetylcholine and activating nicotinic α7 receptors on macrophages. These macrophages in turn release cytokines. Dysfunctions in this pathway have been linked to human inflammatory diseases including rheumatoid arthritis, diabetes and sepsis. Macrophages express the nicotinic α7 receptor and it is likely this receptor that mediates the cholinergic anti-inflammatory response. Therefore, compounds with affinity for the α7nACh receptor on macrophages may be useful for human inflammatory diseases including rheumatoid arthritis, diabetes and sepsis. See, e.g., Czura, et al., J. Intern. Med., 257(2):156-66, 2005; Wang, et al Nature 421:384-388, 2003; de Jonge British Journal of Pharmacology 151:915-929, 2007.

Thus, in accordance with an embodiment of the invention there is provided a method of treating a patient (e.g., a mammal, such as a human) suffering from an inflammatory disease, such as, but not limited to, rheumatoid arthritis, diabetes or sepsis, comprising administering to the patient an effective amount of a compound of formula 1 in a form as described herein.

The mammalian sperm acrosome reaction is an exocytosis process important in fertilization of the ovum by sperm. Activation of an α7 nAChR on the sperm cell has been shown to be essential for the acrosome reaction (Son, J.-H. and Meizel, S. Biol, Reproduct. 68: 1348-1353 2003). Consequently, selective α7 agents demonstrate utility for treating fertility disorders.

In addition, due to their affinity to α7nACh receptors, labeled derivatives of the compounds of formula 1 in a form as described herein, can be used in neuroimaging of the receptors within, e.g., the brain. Thus, using such labeled agents in vivo imaging of the receptors can be performed using, e.g., PET imaging.

The condition of memory impairment is manifested by impairment of the ability to learn new information and/or the inability to recall previously learned information. Memory impairment is a primary symptom of dementia and can also be a symptom associated with such diseases as Alzheimer's disease, schizophrenia, Parkinson's disease, Huntingdon's disease, Pick's disease, Creutzfeldt-Jakob disease, HIV, cardiovascular disease, and head trauma as well as age-related cognitive decline.

Thus, in accordance with an embodiment of the invention there is provided a method of treating a patient suffering from, for example, mild cognitive impairment (MCI), vascular dementia (VaD), age-associated cognitive decline (AACD), amnesia associated w/open-heart-surgery, cardiac arrest, and/or general anesthesia, memory deficits from early exposure of anesthetic agents, sleep deprivation induced cognitive impairment, chronic fatigue syndrome, narcolepsy, AIDS-related dementia, epilepsy-related cognitive impairment, Down's syndrome, Alcoholism related dementia (Korsakoff Syndrome), drug/substance induced memory impairments, Dementia Puglistica (Boxer Syndrome), and animal dementia (e.g., dogs, cats, horses, etc.) comprising administering to the patient an effective amount of a compound of formula 1 in a form as described herein.

In accordance with an embodiment of the invention there is provided a method for improving or stabilizing cognitive function in a subject. In some embodiments, the method is for prevention or treatment of senile dementia, attention deficit disorders, Alzheimer's disease or schizophrenia.

In accordance with an embodiment of the invention there is provided a method of treating a central nervous system (CNS) disease or disorder. In some embodiments, a disease or disorder is selected from the group consisting of psychoses, anxiety, senile dementia, depression, epilepsy, obsessive compulsive disorders, migraine, cognitive disorders, sleep disorders, feeding disorders, anorexia, bulimia, binge eating disorders, panic attacks, disorders resulting from withdrawal from drug abuse, schizophrenia, gastrointestinal disorders, irritable bowel syndrome, memory disorders, Alzheimer's disease, Parkinson's disease, Huntington's chorea, schizophrenia, attention deficit hyperactive disorder, neurodegenerative diseases characterized by impaired neuronal growth, and pain.

### Combination Therapy

Those of ordinary skill in the art will appreciate that compounds of formula 1, in forms as described herein, can be administered in combination with one or more other therapeutically active agents. The phrase “in combination”, as used herein, refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”.

For example, compounds of formula 1, in forms as described herein, may be administered in combination with one or more other modulators of α7 nicotinic acetylcholine receptors. Alternatively or additionally, compounds of formula 1, in forms as described herein, may be administered in combination with one or more other anti-psychotic agents, pain relievers, anti-inflammatories, or other pharmaceutically active agents.

Effective amounts of a wide range of other pharmaceutically active agents are well known to those skilled in the art. However, it is well within the skilled artisan's purview to determine the other pharmaceutically active agent's optimal effective amount range. The compound of formula 1 and the other pharmaceutically active agent can act additively or, in some embodiments, synergistically. In some embodiments of the invention, where another pharmaceutically active agent is administered to an animal, the effective amount of the compound of formula 1 is less than its effective amount would be where the other pharmaceutically active agent is not administered. In this case, without being bound by theory, it is believed that the compound of formula 1 and the other pharmaceutically active agent act synergistically. In some cases, the patient in need of treatment is being treated with one or more other pharmaceutically active agents. In some cases, the patient in need of treatment is being treated with at least two other pharmaceutically active agents.

In some embodiments, the other pharmaceutically active agent is selected from the group consisting of one or more anti-depressant agents, anti-anxiety agents, anti-psychotic agents, or cognitive enhancers. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Suitable selective serotonin reuptake inhibitors include fluoxetine, citolopram, escitalopram, fluvoxamine, paroxetine and sertraline. Examples of monoamine oxidase inhibitors include isocarboxazid, phenelzine, and tranylcypromine. Suitable reversible inhibitors of monoamine oxidase include moclobemide. Suitable serotonin and noradrenaline reuptake inhibitors of use in the present invention include venlafaxine, nefazodone, milnacipran, and duloxetine. Suitable CRF antagonists include those compounds described in International Patent Publication Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in International Patent Publication WO 01/77100.

Anti-anxiety agents that can be used in combination with the compounds of formula 1 include without limitation benzodiazepines and serotonin 1A (5-HT_{1A}) agonists or antagonists, especially 5-HT_{1A} partial agonists, and corticotropin releasing factor (CRF) antagonists. Exemplary suitable benzodiazepines include alprazolam, chlordiazepoxide, clonazepam, chlorazepate, diazepam, halazepam, lorazepam, oxazepam, and prazepam. Exemplary suitable 5-HT_{1A} receptor agonists or antagonists include buspirone, flesinoxan, gepirone and ipsapirone.

Anti-psychotic agents that are used in combination with the compounds of formula 1 include without limitation aliphatic phethiazine, a piperazine phenothiazine, a butyrophenone, a substituted benzamide, and a thioxanthine. Additional examples of such drugs include without limitation haloperidol, olanzapine, clozapine, risperidone, pimozide, aripiprazol, and ziprasidone. In some cases, the drug is an anticonvulsant, e.g., phenobarbital, phenyloin, primidone, or carbamazepine.

Cognitive enhancers that are used in combination with the compounds of formula 1 include, without limitation, drugs that modulate neurotransmitter levels (e.g., acetylcholinesterase or cholinesterase inhibitors, cholinergic receptor agonists or serotonin receptor antagonists), drugs that modulate the level of soluble Aβ, amyloid fibril formation, or amyloid plaque burden (e.g., γ-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs that are co-administered with the compounds of the invention include cholinesterase inhibitors, (e.g., tacrine (COGNEX®), donepezil (ARICEPT®), rivastigmine (EXELON®) galantamine (REMINYL®), metrifonate, physostigmine, and Huperzine A), N-methyl-D-aspartate (NMDA) antagonists and agonists (e.g., dextromethorphan, memantine, dizocilpine maleate (MK-801), xenon, remacemide, eliprodil, amantadine, D-cycloserine, felbamate, ifenprodil, CP-101606 (Pfizer), Delucemine, and compounds described in U.S. Pat. Nos. 6,821,985 and 6,635,270), ampakines (e.g., cyclothiazide, aniracetam, CX-516 (Ampalex®), CX-717, CX-516, CX-614, and CX-691 (Cortex Pharmaceuticals, Inc. Irvine, Calif.), 7-chloro-3-methyl-3-4-dihydro-2H-1,2,4-benzothiadiazine S,S-dioxide (see Zivkovic et al., 1995, J. Pharmacol. Exp. Therap., 272:300-309; Thompson et al., 1995, Proc. Natl. Acad. Sci. USA, 92:7667-7671), 3-bicyclo[2,2,1]hept-5-en-2-yl-6-chloro-3,4-dihydro-2H-1,2,4-benzothiadiazine-7-sulfonamide-1,1-dioxide (Yamada, et al., 1993, J. Neurosc. 13:3904-3915); 7-fluoro-3-methyl-5-ethyl-1,2,4-benzothiadiazine-S,S-dioxide; and compounds described in U.S. Pat. No. 6,620,808 and International Patent Application Nos. WO 94/02475, WO 96/38414, WO 97/36907, WO 99/51240, and WO 99/42456), benzodiazepine (BZD)/GABA receptor complex modulators (e.g., progabide, gengabine, zaleplon, and compounds described in U.S. Pat. Nos. 5,538,956, 5,260,331, and 5,422,355); serotonin antagonists (e.g., 5HT receptor modulators, 5HT_{1A} antagonists or agonists (including without limitation lecozotan and compounds described in U.S. Pat. Nos. 6,465,482, 6,127,357, 6,469,007, and 6,586,436, and in PCT Publication No. WO 97/03982) and 5-HT₆ antagonists (including without limitation compounds described in U.S. Pat. Nos. 6,727,236, 6,825,212, 6,995,176, and 7,041,695)); nicotinics (e.g., niacin); muscarinics (e.g., xanomeline, CDD-0102, cevimeline, talsaclidine, oxybutin, tolterodine, propiverine, tropsium chloride and darifenacin); monoamine oxidase type B (MAO B) inhibitors (e.g., rasagiline, selegiline, deprenyl, lazabemide, safinamide, clorgyline, pargyline, N-(2-aminoethyl)-4-chlorobenzamide hydrochloride, and N-(2-aminoethyl)-5(3-fluorophenyl)-4-thiazolecarboxamide hydrochloride); phosphodiesterase (PDE) IV inhibitors (e.g., roflumilast, arofylline, cilomilast, rolipram, RO-20-1724, theophylline, denbufylline, ARIFLO, ROFLUMILAST, CDP-840 (a tri-aryl ethane) CP80633 (a pyrimidone), RP 73401 (Rhone-Poulenc Rorer), denbufylline (SmithKline Beecham), arofylline (Almirall), CP-77,059 (Pfizer), pyrid[2,3d]pyridazin-5-ones (Syntex), EP-685479 (Bayer), T-440 (Tanabe Seiyaku), and SDZ-ISQ-844 (Novartis)); G proteins; channel modulators; immunotherapeutics (e.g., compounds described in U.S. Patent Application Publication No. US 2005/0197356 and US 2005/0197379); anti-amyloid or amyloid lowering agents (e.g., bapineuzumab and compounds described in U.S. Pat. No. 6,878,742 or U.S. Patent Application Publication Nos. US 2005/0282825 or US 2005/0282826); statins and peroxisome proliferators activated receptor (PPARS) modulators (e.g., gemfibrozil (LOPID®), fenofibrate (TRICOR®), rosiglitazone maleate (AVANDIA®), pioglitazone (Actos™), rosiglitazone (Avandia™), clofibrate and bezafibrate); cysteinyl protease inhibitors; an inhibitor of receptor for advanced glycation endproduct (RAGE) (e.g., aminoguanidine, pyridoxaminem carnosine, phenazinediamine, OPB-9195, and tenilsetam); direct or indirect neurotropic agents (e.g., Cerebrolysin, piracetam, oxiracetam, AIT-082 (Emilieu, 2000, Arch. Neurol. 57:454)); beta-secretase (BACE) inhibitors, α-secretase, immunophilins, caspase-3 inhibitors, Src kinase inhibitors, tissue plasminogen activator (TPA) activators, AMPA (alpha-amino-3-hydroxy-5-methyl-4-isoxazolepropionic acid) modulators, M4 agonists, JNK3 inhibitors, LXR agonists, H3 antagonists, and angiotensin IV antagonists. Other cognition enhancers include, without limitation, acetyl-1-carnitine, citicholine, huperzine, DMAE (dimethylaminoethanol), Bacopa monneiri extract, Sage extract, L-alpha glyceryl phosphoryl choline, Ginko biloba and Ginko biloba extract, Vinpocetine, DHA, nootropics including Phenyltropin, Pikatropin (from Creative Compounds, LLC, Scott City, Mo.), besipirdine, linopirdine, sibopirdine, estrogen and estrogenic compounds, idebenone, T-588 (Toyama Chemical, Japan), and FK960 (Fujisawa Pharmaceutical Co. Ltd.). Compounds described in U.S. Pat. Nos. 5,219,857, 4,904,658, 4,624,954 and 4,665,183 are also useful as cognitive enhancers as described herein. Cognitive enhancers that act through one or more of the above mechanisms are also within the scope of this invention.

In some embodiments, the compound of formula 1 and cognitive enhancer act additively or, in some embodiments, synergistically. In some embodiments, where a cognitive enhancer and a compound of formula 1 are co-administered to an animal, the effective amount of the compound or pharmaceutically acceptable salt of the compound of the invention is less than its effective amount would be where the cognitive enhancer agent is not administered. In some embodiments, where a cognitive enhancer and a compound of formula 1 are co-administered to an animal, the effective amount of the cognitive enhancer is less than its effective amount would be where the compound or pharmaceutically acceptable salt of the invention is not administered. In some embodiments, a cognitive enhancer and a compound of formula 1 are co-administered to an animal in doses that are less than their effective amounts would be where they were no co-administered. In these cases, without being bound by theory, it is believed that the compound of formula 1 and the cognitive enhancer act synergistically.

In some embodiments, the other pharmaceutically active agent is an agent useful for treating Alzheimer's disease or conditions associate with Alzheimer's disease, such as dementia. Exemplary agents useful for treating Alzheimer's disease include, without limitation, donepezil, rivastigmine, galantamine, memantine, and tacrine.

In some embodiments, the compound of formula 1 is administered together with another pharmaceutically active agent in a single administration or composition.

In some embodiments, a composition comprising an effective amount of the compound of formula 1 and an effective amount of another pharmaceutically active agent within the same composition can be administered.

In another embodiment, a composition comprising an effective amount of the compound of formula 1 and a separate composition comprising an effective amount of another pharmaceutically active agent can be concurrently administered. In another embodiment, an effective amount of the compound of formula 1 is administered prior to or subsequent to administration of an effective amount of another pharmaceutically active agent. In this embodiment, the compound of formula 1 is administered while the other pharmaceutically active agent exerts its therapeutic effect, or the other pharmaceutically active agent is administered while the compound of formula 1 exerts its preventative or therapeutic effect.

Thus, in some embodiments, the invention provides a composition comprising an effective amount of the compound of formula 1 and a pharmaceutically acceptable carrier. In some embodiments, the composition further comprises a second pharmaceutically active agent.

In another embodiment, the composition further comprises a pharmaceutically active agent selected from the group consisting of one or more other antidepressants, anti-anxiety agents, anti-psychotic agents or cognitive enhancers. Antidepressants, anti-anxiety agents, anti-psychotic agents and cognitive enhancers suitable for use in the composition include the antidepressants, anti-anxiety agents, anti-psychotic agents and cognitive enhancers provided above.

In another embodiment, the pharmaceutically acceptable carrier is suitable for oral administration and the composition comprises an oral dosage form.

In some embodiments, one or more compounds of formula 1 is administered in combination with antidepressant drug treatment, antipsychotic drug treatment, and/or anticonvulsant drug treatment.

In certain embodiments, a compound of formula 1 is administered in combination with one or more selective serotonin reuptake inhibitors (SSRIs) (for example, fluoxetine, citalopram, escitalopram oxalate, fluvoxamine maleate, paroxetine, or sertraline), tricyclic antidepressants (for example, desipramine, amitriptyline, amoxipine, clomipramine, doxepin, imipramine, nortriptyline, protriptyline, trimipramine, dothiepin, butriptyline, iprindole, or lofepramine), aminoketone class compounds (for example, bupropion); in some embodiments, a compound of formula 1 is administered in combination with a monoamine oxidase inhibitor (MAOI) (for example, phenelzine, isocarboxazid, or tranylcypromine), a serotonin and norepinepherine reuptake inhibitor (SNRI) (for example, venlafaxine, nefazodone, milnacipran, duloxetine), a norepinephrine reuptake inhibitor (NRI) (for example, reboxetine), a partial 5-HT_{1A} agonist (for example, buspirone), a 5-HT_{2A} receptor antagonist (for example, nefazodone), a typical antipsychotic drug, or an atypical antipsychotic drug. Examples of such antipsychotic drugs include aliphatic phethiazine, a piperazine phenothiazine, a butyrophenone, a substituted benzamide, and a thioxanthine. Additional examples of such drugs include haloperidol, olanzapine, clozapine, risperidone, pimozide, aripiprazol, and ziprasidone. In some cases, the drug is an anticonvulsant, e.g., phenobarbital, phenyloin, primidone, or carbamazepine. In some cases, the compound of formula 1 is administered in combination with at least two drugs that are antidepressant drugs, antipsychotic drugs, anticonvulsant drugs, or a combination thereof.

### EXEMPLIFICATION

### Example 1

### Attempts to Obtain a Solid Form of the Free Base of a Compound of Formula 1

Attempts were made to crystalline the free base form of a compound of formula 1 from a variety of solvents, including, for example, IPA, MeOH, EtOH, CH₃CN, EtOAc, CH₂Cl₂, CHCl₃, 1,2-dichloroethane, THF, iPOAc; in each gave an oily product was obtained.

### Example 2

### Synthesis of a Compound of Formula 1

### Example 2A

### 5-(4-Acetyl-[1,4]diazepan-1-yl)-pentanoic acid [5-(4-methoxy-phenyl)-1H-pyrazol-3-yl]-amide

i) 5-Bromo-pentanoic acid [5-(4-methoxy-phenyl)-1H-pyrazol-3-yl]-amide

A solution of 5-bromovaleryl chloride (2.1 mL, 15.7 mmol, 1 eq) in dry DMA (35 mL) was cooled to −10° C. (ice/water bath) under N₂; a solution of 5-(4-methoxy-phenyl)-1H-pyrazol-3-ylamine (3.0 g, 15.7 mmol, 1 equiv.) and diisopropylethylamine (2.74 mL, 15.7 mmol, 1 equiv.) in dry DMA (15 mL) was added over 30 min. After 2 hrs at −10° C., LC-MS shows completion of the reaction which was quenched by addition of H₂O (ca. 50 mL). The solid which precipitates was filtered and washed with Et₂O, to give 4.68 g of 5-bromo-pentanoic acid [5-(4-methoxy-phenyl)-1H-pyrazol-3-yl]-amide as a white powder (13.3 mmol, 85% yield).

mp=149.5-151.5° C.

C₁₅H₁₈BrN₃O₂ Mass (calculated) [352.23]; (found) [M+H⁺]=352.09/354.10

LC Rt=2.07, 95% (5 min method)

¹H-NMR (400 MHz, DMSO-d₆): δ 1.69-1.63 (2H, m); 1.81-1.75 (2H, m); 2.29 (2H, t); 3.52 (2H, t); 3.75 (3H, s); 6.75 (1H, bs); 6.96 (2H, d); 7.6 (2H, d); 10.28 (1H, s); 12.57 (1H, s)

ii) 5-(4-Acetyl-[/, 4]diazepan-1-yl)-pentanoic acid [5-(4-methoxy-phenyl)-1H-pyrazol-3-yl]-amide

To 750 mg (1.96 mmol) of 5-bromo-pentanoic acid [5-(4-methoxy-phenyl)-2H-pyrazol-3-yl]-amide in 7 mL of DMA, N-acetyl-diazepine (278 mg, 1.96 mmol) and NaI (240 mg, 1.96 mmol) were added and the reaction heated at 60° C. for 18 hours. Upon complete conversion (as monitored by LC-MS) the mixture was diluted with 20 mL of DCM and washed with water. The organic phase was concentrated under reduced pressure to afford a residue which was purified with a SiO₂ column (10 g) eluting with a gradient from DCM to MeOH 90:10. The title compound (380 mg) was recovered pure (yield 46%). C₂₂H₃₁N₅O₃ Mass (calculated) [413]; (found) [M+H^{+]=414}

LC Rt=1.91, 100% (10 min method)

¹H-NMR (400 MHz, DMSO-d₆): δ 1.53-1.75 (4H, m), 1.90-2.15 (5H, m), 2.28-2.42 (2H, m), 2.90-3.26 (3H, m), 3.34-3.58 (3H, m), 3.71-3.88 (7H, m)

### Example 2B

### 5-(4-acetyl-1,4-diazepan-1-yl)-N-(5-(4-methoxyphenyl)-1H-pyrazol-3-yl)pentanamide (mono Hydrochloride Salt)

To a solution of 5-(4-methoxyphenyl)-1H-pyrazol-3-ylamine (12 g, 62.8 mmol) and N,N-diisopropylethylamine (10.96 mL, 62.8 mmol) in dry N,N-dimethylformamide (150 mL) at −10° C. was added a solution of 5-bromovaleryl chloride (8.4 mL, 62.8 mmol) in dry N,N-dimethylformamide (50 mL) slowly (˜40 min) and the reaction mixture was allowed to stir at −10 to 0° C. for 8 hrs. Sodium iodide (9.44 g, 62.8 mmol) was added at 0° C. and followed by N-acetylhomopiperazine (8.24 mL, 62.8 mmol) and N,N-diisopropylethylamine (10.96 mL, 62.8 mmol) and the reaction mixture was allowed to stir at 50° C. for 18 hrs. The solvent was removed in vacuo. The residue was dissolved in methylene chloride (500 mL) and saturated aqueous sodium bicarbonate (500 mL) and the mixture was stirred at room temperature for 30 minutes. The organic layer was separated, dried over sodium sulfate, and the solvent was removed in vacuo to provide 25.8 g (99%) of 5-(4-acetyl-1,4-diazepan-1-yl)-N-(5-(4-methoxyphenyl)-1H-pyrazol-3-yl)pentanamide as a thick light yellow oil (crude).

Then to a solution of the crude 5-(4-acetyl-1,4-diazepan-1-yl)-N-(5-(4-methoxyphenyl)-1H-pyrazol-3-yl)pentanamide (as a free base) in methylene chloride (270 mL) at room temperature was added hydrogen chloride (65 mL, 1.0 M in ethyl ether) slowly. The resulting suspension was allowed to stir at room temperature for 1 hour. The solvent was removed in vacuo to afford 33 g as a yellow foam, mono hydrochloride salt. The foam was dissolved in solvents (330 mL, acetonitrile:methanol=33:1) at 60-70° C. and a crystal seed was added. The mixture was slowly cooled down to the room temperature and allowed to stir at room temperature for 15 hours. The resulting precipitate was filtered and dried to give 20.5 g (72%) of the title compound as a white crystal, mono hydrochloride salt. MS [M−H]⁻ m/z 412.3; mp. 132-133° C. ¹H NMR (400 MHz, DMSO-d₆) δ 10.6-10.8 (br, 1H), 10.45 (s, 1H), 7.64 (d, J=8 Hz, 2H), 7.00 (d, J=8 Hz, 2H), 6.74 (s, H), 4.00 (m, 1H), 3.77 (s, 3H), 3.4-3.6 (m, 6H), 2.9˜3.0 (m, 5H), 2.34 (m, 2H), 2.0 (s, 3H), 1.65-1.70 (m, 2H), 1.55-1.65 (m, 2H).

### Example 2C

### 5-(4-acetyl-1,4-diazepan-1-yl)-N-(5-(4-methoxyphenyl)-1H-pyrazol-3-yl)pentanamide (mono Hydrochloride Salt)

i) 3-(4-methoxyphenyl)-3-oxopropanenitrile

A solution of methyl p-anisate in acetonitrile was cooled to −10° C. Lithium bis(trimethylsilyl)amide (1 M in THF) was added dropwise over a minimum of 3 hr. The mixture was held at −10 to 0° C. until reaction completion. The reaction mixture was quenched with water and the pH adjusted to 3-4 with conc HCl. The mixture was stirred for 1 hr. The product was isolated by filtration, washed with water and dried in a vacuum oven. The yield was 73%.

ii) 5-(4-methoxyphenyl)-1H-pyrazol-3-amine

A suspension of 3-(4-methoxyphenyl)-3-oxopropanenitrile in ethanol was heated to 60° C. Hydrazine hydrate was added dropwise over a minimum of 30 min at 60° C. The resulting solution was held at 60° C. until reaction completion, generally 15-18 hr. The reaction mixture was quenched with water. Ethanol was removed by distillation to about 5 volumes. The product was isolated by filtration, washed with water and dried in a vacuum oven. The yield was 88-95%.

iii) 5-bromo-N-(5-(4-methoxyphenyl)-1H-pyrazol-3-yl)pentanamide

A solution of 5-(4-methoxyphenyl)-1H-pyrazol-3-amine and diisopropylethylamine in 10 volumes of a 9:1 mixture of acetonitrile:DMF was cooled to −10° C. 5-Bromovaleryl chloride was added dropwise over a minimum of 3 hr at −10° C. The resulting solution was held at −10° C. until reaction completion, generally 2 hr. The reaction mixture was quenched with water. The product was isolated by filtration, washed with water, TBME and suction dried. The product-wet cake was purified by re-slurrying in TBME at 35° C. for a minimum of 2 hr. The yield was 70-80%.

iv)₅-(4-acetyl-1,4-diazepan-1-yl)-N-(5-(4-methoxyphenyl)-1H-pyrazol-3-yl)pentanamide Hydrochloride

Bromopyrazole is mixed with K₂CO₃ and KI in 10 volumes of acetone at room temperature and N-acetylhomopiperazine was added over 1 hr. The reaction mixture was stirred until the reaction was complete. The mixture was filtered, removing the inorganics, washed with acetone and distilled to 2 volumes. The freebase was extracted into methyl THF/EtOH and washed with NaCl and NaHCO₃. The solvent was replaced with EtOH, a strength of the solution was determined, and 0.93 eq of HCl based on the available freebase was added to a mixture of acetone, ethanol and water. Careful monitoring of the pH yielded crystalline product in a 70% overall yield and the desired form 1.

### Example 2D

### 5-(4-acetyl-1,4-diazepan-1-yl)-N-(5-(4-methoxyphenyl)-1H-pyrazol-3-yl)pentanamide (mono hydrochloride salt)

i) 5-(4-methoxy-phenyl)-1H-pyrazol-3-ylamine

The intermediate 5-(4-methoxy-phenyl)-1H-pyrazol-3-ylamine is commercially available from Sigma-Alrich (USA), but can be made using the following general procedure:

Aryl β-ketonitrile Synthesis

To a solution of an aromatic ester (6.5 mmol) in dry toluene (6 mL), under N₂, NaH (50-60% dispersion in mineral oil, 624 mg, 13 mmol) was carefully added. The mixture was heated at 80° C. and then dry CH₃CN was added dropwise (1.6 mL, 30.8 mmol). The reaction was heated for 18 h and generally the product precipitated from the reaction mixture as a salt. The reaction was allowed to cool to room temperature and the solid formed was filtered and then dissolved in water. The solution was acidified with 2 N HCl solution, and upon reaching a pH between 2-4, the product precipitated and was filtered. If no precipitation occurred, the product was extracted with DCM. After aqueous workup, the products were generally pure enough to be used in the next step without further purification. The isolated yield was generally 40-80%.

Aryl Aminopyrazole Synthesis

To a solution of β-ketonitrile (7.5 mmol) in absolute EtOH (15 mL), hydrazine monohydrate (0.44 mL, 9.0 mmol) was added and the reaction was heated at reflux for 18 hrs. The reaction mixture was allowed to cool to room temperature and the solvent was evaporated under reduced pressure. The residue was dissolved in 20 mL of DCM and washed with water. The organic phase was concentrated to give a crude product that was purified by SiO₂ column or by precipitation from Et₂O. For example, the 2-methoxy derivative was purified by SiO₂ chromatography, eluting with a DCM/MeOH gradient (from 100% DCM to 90/10 DCM/MeOH); the 3-methoxy derivative was triturated with Et₂O. Yields were generally 65-90%.

ii) 5-bromo-pentanoic acid [5-(4-methoxy-phenyl)-1H-pyrazol-3-yl]amide

A solution of 5-bromovaleryl chloride (2.1 mL, 15.7 mmol) in dry dimethylacetamide (DMA) (35 mL) was cooled to −10° C. (ice water bath) under N₂; a solution of 5-(4-methoxy-phenyl)-1H-pyrazol-3-ylamine (3.0 g, 15.7 mmol) and diisopropylethylamine (2.74 mL, 15.7 mmol) in dry DMA (15 mL) was added over 30 min. After two hours at −10° C., LCMS shows completion of the reaction (acylation on the pyrazole ring was also detected). The reaction was quenched by addition of H₂O (ca. 50 mL), and the thick white precipitate formed upon addition of water was recovered by filtration. When the reaction was allowed to reach room temperature before quenching, a putative exchange of Br with Cl caused reactivity problems in subsequent steps. Washing with Et₂O (3×10 mL) efficiently removed the byproduct (acylation on pyrazole ring). 4.68 g of the title compound was obtained as a white powder (13.3 mmol, 85% yield). Mp=149.5-151.5° C.

iii) 5-(4-acetyl-[1,4]diazepan-1-yl)-pentanoic acid [5-(4-methoxy-phenyl)-1H-pyrazol-3-yl]-amide

5-bromo-pentanoic acid [5-(4-methoxy-phenyl)-1H-pyrazol-3-yl]amide (1.5 g, 4.26 mmol) was dissolved in DMF (15 mL), and sodium iodide (0.64 g, 4.26 mmol) was added followed by N-acetylhomopiperazine (0.56 mL, 4.26 mmol) and diisopropylethylamine (0.74 mL, 4.26 mmol). The reaction was stirred under N₂ at 50° C. for 18 hrs. Upon reaction completion (as monitored by LCMS), the solvent was removed at reduced pressure and the resulting oily residue was dissolved in DCM (20 mL), washed with sat. Na₂CO₃ (2×20 mL) and sat. NaCl (2×20 mL), and dried over Na₂SO₄. Upon solvent removal, 1.7 g of crude product as a thick oil were obtained. The product was purified by SiO₂ chromatography (10 g cartridge-flash SI II from IST) employing DCM and DCM:MeOH 9:1 to yield 0.92 g of pure product and 0.52 g of less pure product. A second purification of the impure fractions using a 5 g SiO₂ cartridge was performed using the same eluent. Overall, 1.09 g of 5-(4-acetyl-[1,4]diazepan-1-yl)-pentanoic acid [5-(4-methoxy-phenyl)-1H-pyrazol-3-yl]-amide were obtained (2.64 mmol, 62% yield) as a thick light yellow oil. MS (ES+): 414.26 (M+H)⁺.

iv)₅-(4-acetyl-1,4-diazepan-1-yl)-N-(5-(4-methoxyphenyl)-1H-pyrazol-3-yl)pentanamide (Mono Hydrochloride Salt)

5-(4-acetyl-[1,4]diazepan-1-yl)-pentanoic acid [5-(4-methoxy-phenyl)-1H-pyrazol-3-yl]-amide (1.05 g, 2.54 mmol) was dissolved in a minimum amount of DCM (5 mL) and cooled to 0° C. HCl (2.0 M in Et₂₀, 1.4 mL, 2.89 mmol) was added and the mixture stirred at rt until precipitation of the salt was complete (about 10 min.). The solid was filtered, washed with Et₂O several times, and dried in a dessicator to yield 1.09 g of the hydrochloride salt (2.42 mmol, 95% yield). Melting point was not determined due to the extreme hygroscopicity of the sample. MS (ES+): 414.26 (M+H)⁺.

### Example 2E

### 5-(4-acetyl-1,4-diazepan-1-yl)-N-(5-(4-methoxyphenyl)-1H-pyrazol-3-yl)pentanamide (Mono Hydrochloride Salt)

i)₅-(4-acetyl-[, 4]diazepan-1-yl)-N-[5-(4-methoxy-phenyl)-1H-pyrazol-3-yl]-pentanamide

To a cylindrical, jacketed 3 L reactor equipped with nitrogen inerting, agitator, condenser/distillation head, and temperature control, 5-bromo-pentanoic acid [5-(4-methoxy-phenyl)-1H-pyrazol-3-yl]amide (0.15 kg, 0.426 mol), potassium carbonate (0.059 kg, 0.426 mol), potassium iodide (0.071 kg, 0.426 mol), and acetone (1.18 kg, 1.5 L) were added (at 20° C.) to form a white mixture. The mixture was stirred (235 rpm) at 25-30° C. for a minimum of 15 min. N-acetylhomopiperazine (0.062 kg, 0.057 L, 0.434 mol) was added via addition funnel to the reactor over a minimum of 45 min., maintaining the temperature in the range of 25-30° C. The addition funnel was rinsed with 0.05 L acetone. A white mixture persisted. The mixture was stirred (235 rpm) in the range of 25-30° C. for a minimum of 16 h, forming a white/yellow mixture. The reaction progress was monitored by HPLC and was considered complete when there was ≦2% of the starting material (bromopyrazole) and ≦2% of the iodopyrazole present.

The reactor contents were cooled to 5-15° C. over a minimum of 15 min with agitation (295 rpm) to form a white/yellow mixture that was then stirred for a minimum of 1 h. To remove inorganics, the mixture was then filtered on a Buchner funnel with filter paper using house vacuum for 1.5 min. The cake was washed twice with acetone (total of 0.24 kg, 0.30 L) at 5-15° C. The wash was combined with the mother liquor from the prior filtration and used to rinse the reactor. The filtrate was concentrated to a volume of approximately 0.45 L to form a clear solution.

ii) Aqueous Workup

To a reactor containing the material from step i, 1.5 L of a freshly made homogeneous solution of methyl THF (1.22 kg, 1.42 L) and ethanol (0.059 kg, 0.075 L) was added at 25° C., forming a hazy solution. To this, 0.45 L of a 5% solution of sodium chloride (0.022 kg) in water (0.43 L) was added at 25° C. The resulting mixture was heated with stirring to 30-35° C. over a minimum of 15 min., forming a clear biphasic solution. The agitation was stopped to allow the layers to settle, the product being in the upper layer. The layers were separated, keeping any emulsion in the upper organic layer. The organic layer was retained. A homogeneous 5% solution of sodium bicarbonate (0.03 kg) in water (0.57 L) at 25° C. was used to wash organic layer, stirring for a minimum of 5 min. at 10-15° C. The agitation was stopped to allow the layers to settle, the product being in the upper layer. The layers were separated, keeping any emulsion in the upper organic layer. The organic layer was retained and concentrated to a volume of 0.35 L, forming a hazy solution. The mixture was chased with ethanol to remove residual water.

iii) 5-(4-acetyl-[1,4]diazepan-1-yl)-N-[5-(4-methoxy-phenyl)-1H-pyrazol-3-yl]-pentanamide HCl

To a reactor containing the material from step ii, 0.47 kg (0.60 L) of acetone was added. The resulting mixture was heated with stirring to 25-30° C. over a minimum of 10 min., forming a hazy solution. The contents of the reactor were clarified through a polypropylene pad into a tared 2 L suction flask using vacuum, maintaining the contents of the reactor at 25-30° C. Suction was maintained until filtration stopped. The reactor and filter pad were rinsed with acetone (0.05 L) at 20-25° C. The filtrates from the suction flask were transferred to the reactor and rinsed using acetone (0.05 L). A solution of 5% HCl (0.042 kg, 0.036 L) in acetone (0.174 L) and alcohol solution (0.0174 L of ethanol:acetone (91:9) v/v) was prepared and stirred until homogeneous at 10° C. To the reactor, 0.05 L of water was added to form a clear solution. One third of the 5% HCl solution (0.076 L) was added to the reactor over a minimum of 20 min., maintaining the temperature in the range of 20-25° C. A second third of the 5% HCl solution (0.076 L) was then added to the reactor over a minimum of 20 min., maintaining the temperature in the range of 20-25° C. The contents of the reactor were seeded with 75 mg of 5-(4-acetyl-[1,4]diazepan-1-yl)-N-[5-(4-methoxy-phenyl)-1H-pyrazol-3-yl]-pentanamide HCl (e.g., Form 1), followed by the addition of the last third of 5% HCl solution (0.076 L) over a minimum of 20 min., maintaining the temperature in the range of 20-25° C. Another 0.08 equiv. of the 5% HCl solution (0.023 L) was then added to the reactor over a minimum of 30 min., maintaining the temperature in the range of 20-25° C. Judicious monitoring of pH was performed to attain the desired pH range of 5.2-5.8.

The mixture was stirred at 20-25° C. for a minimum of 1 hr., forming a thin suspension. Acetone (0.6 L) was added over a minimum of 60 min., maintaining the temperature in the range of 20-25° C. The mixture was stirred at 20-25° C. for a minimum of 60 min. Acetone (1.5 L) was added to the reactor over a minimum of 3 hr., maintaining the temperature in the range of 20-25° C., forming a thick suspension. The mixture was then stirred at 20-25° C. for a minimum of 12 h. Crystallization was considered complete when there was ≦20% of the product present in the mother liquor.

The mixture was then filtered on a Buchner funnel (polypropylene pad) using house vacuum. A solution of water (0.009 L), acetone (0.23 L) and 0.06 L alcohol (ethanol:acetone (91:9) v/v) was stirred until homogeneous (20% ethanol, 3% water, 77% acetone overall). This solution was used to wash the filter cake twice (0.15 L×2). A solution of water (0.009 L), acetone (0.171 L) and 0.12 L alcohol (ethanol:acetone (91:9) v/v) was stirred until homogeneous (40% ethanol, 3% water, 57% acetone overall). This solution was used to wash the filter cake (0.30 L). The wet cake was subjected to suction under nitrogen using house vacuum and held for 30 min. after dripping stopped. Product purity was checked by HPLC and additional washing was performed if total impurities were not ≦2%. Product was oven dried in a vacuum oven with nitrogen bleed at 38-45° C., maintaining vacuum at 20 torr for a minimum of 12 h until loss on drying of less than 1% was obtained. Following drying, 0.119 kg of the title compound was obtained in 62% yield (67% adjusted for aliquots removed during process; 60% when corrected for strength or purity). Melting point=185° C.; crystal form=form 1; particle size=D90<89.4 um, D50<19.2 um.

### Example 3

### Preparation of Salt Forms of a Compound of Formula 1

The present Example describes the preparation of salt forms of a compound of formula 1, including acetic, citric, D-glucuronic, fumaric, hydrochloric, oxalic, maleic, phosphoric, salicylic, succinic, sulfuric, and tartaric acid forms.

As shown in Table 1, no solid was observed for acetic and tartaric acid forms; a sticky gel was observed with citric, maleic, oxalic, salicylic, and succinic acid salt forms. Solid forms were observed for fumaric, hydrochloric, phosphoric, and sulfuric acid salt forms. The fumaric acid salt form appeared to form a solvate. The phosphoric and sulfuric acid salt forms oiled out and crystallized overnight. The D-glucoronic acid salt form achieved an amorphous-like solid. By surprising contrast, the hydrochloric acid salt form readily adopted a solid form. Indeed, at least four different crystalline forms (i.e., polymorphs) were observed for the hydrochloric acid salt form (see Example 4, below).

TABLE 1

| Counter Ion Used | Solid Obtained | Melting Onset | Hygroscopicity |
|---|---|---|---|
| Acetic acid | No solid | | |
| Citric acid | Sticky gel | | |
| D-glucoronic acid | Amorphous-like | | |
| | solid (a few small | | |
| | peaks) | | |
| Fumaric acid | Solvate | 110° C. desol | |
| | | 136° C. | |
| Hydrochloric acid | Crystalline solid | 185° C. | No |
| | | 165° C. | Somewhat |
| | | 125° C. | Yes |
| | | 125° C. | Not measured |
| | | three peaks: | Yes |
| | | about 100 | |
| | | about 180; and | |
| | | about 200° C. | |
| Maleic acid | Sticky gel | | |
| Oxalic acid | Sticky gel | | |
| Phosphoric acid | Oiled out and | 134° C. | Starts at 10% and |
| | crystallized in | 2% weight loss | holds about 2% |
| | seven days to | in TGA | water at 50% RH |
| | crystalline solid | | |
| Salicylic acid | Sticky gel | | |
| Succinic acid | Sticky gel | | |
| Sulfuric acid | Oiled out and | 119° C. | At 50% RH |
| | crystallized | 3% weight loss | maintains 0.4% |
| | overnight to | in TGA | water |
| | crystalline solid | | |
| Tartaric acid | No solid | | |

Differential scanning calorimetry data were collected for each solid form achieved using a DSC (TA instruments, model Q1000) under the following parameters: 50 mL/min purge gas (N₂); scan range 40 to 200° C., scan rate 10° C./min.

Thermogravimetric analysis data were collected using a TGA instruments (Mettler Toledo, model TGA/SDTA 851e) under the following parameters: 40 ml/min purge gas (N₂); scan range 30 to 250° C., scan rate 10° C./min.

X-ray data were acquired using an X-ray powder diffractometer (Bruker-axs, model D8 advance) having the following parameters: voltage 40 kV, current 40.0 mA, scan range 5 to 30°, scan step size 0.01°, total scan time 33 minutes, VANTEC detector, and antiscattering slit 1 mm.

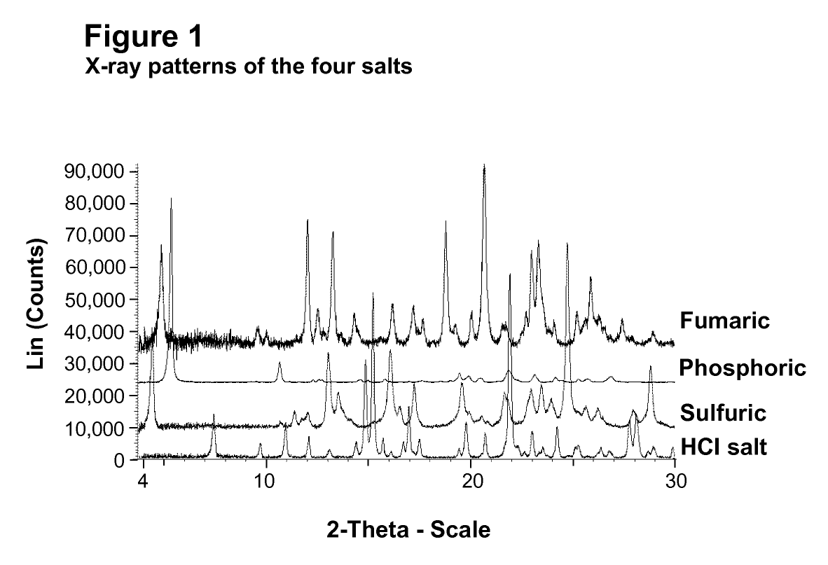

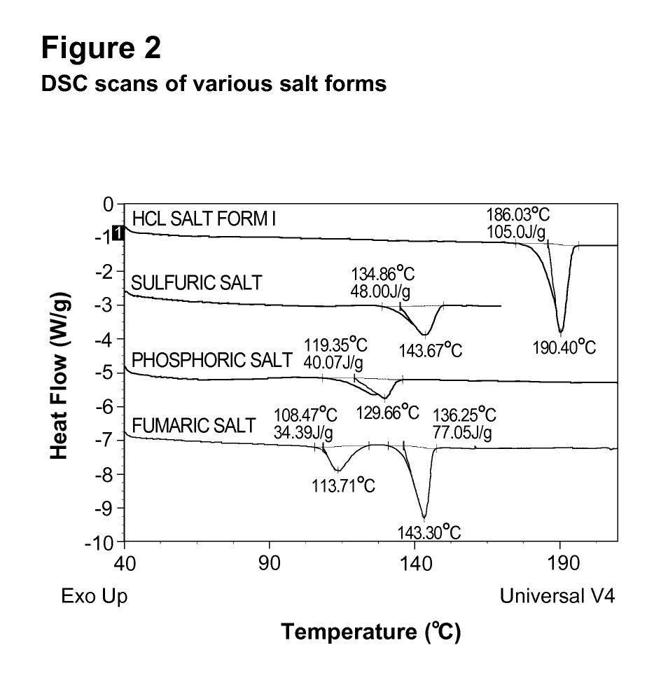

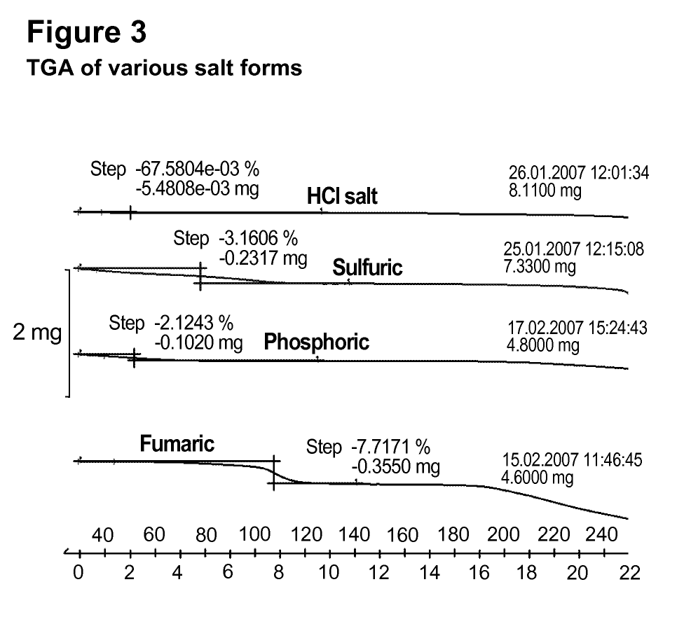

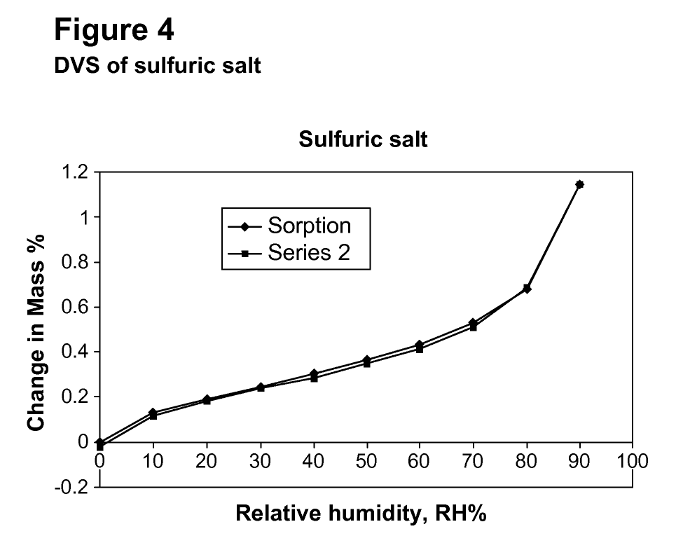

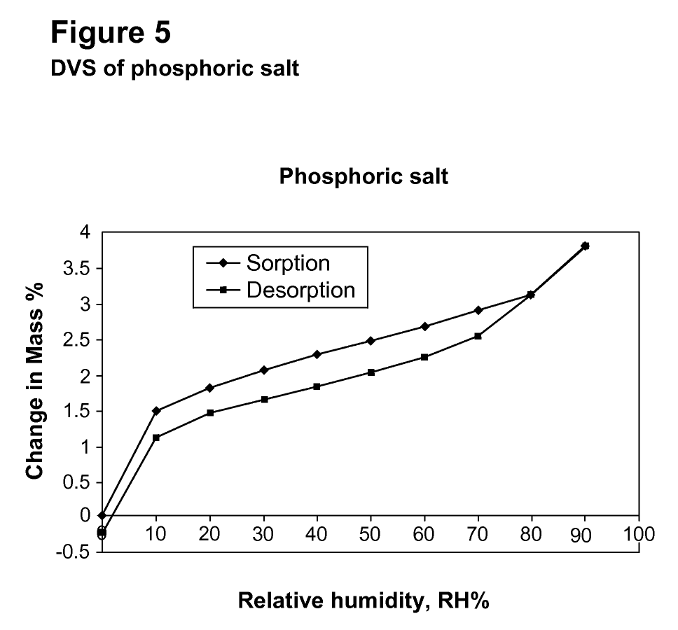

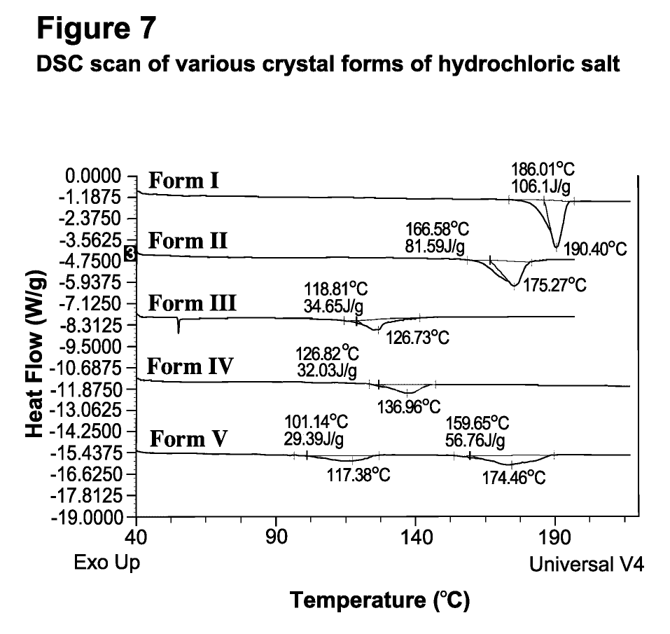

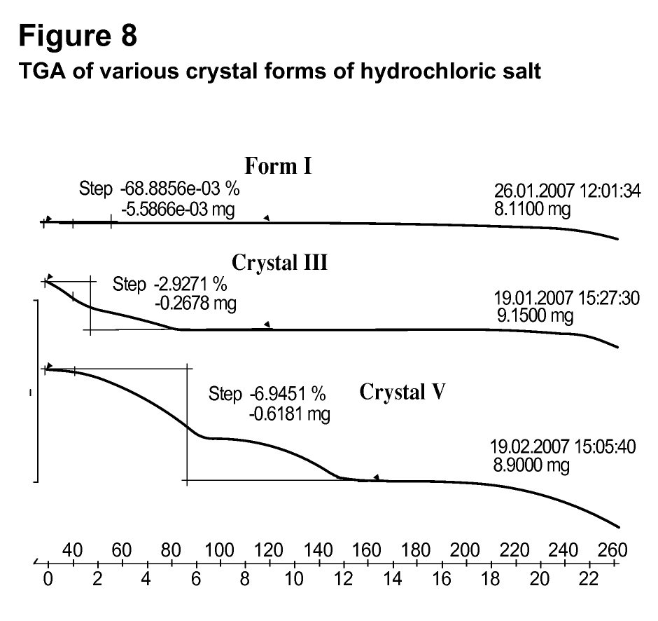

Salt forms of a compound of formula 1 were investigated with X-ray powder diffraction, DSC, TGA and microscopy. FIG. 1 illustrates X-ray patterns observed for various salt forms. FIGS. 2 and 3 present results of thermal studies performed on such salt forms. FIGS. 4 and 5 present DVS data of sulfuric and phosphoric salts. The sulfate salt form shows a low degree of hygroscopicity (about 0.4% water at 50% RH); the phosphate salt form absorbed more water (about 2% water at 50% RH). FIGS. 6-12 show characterization data for hydrochloride salt forms.

As can be seen, the sulfate salt adopted a crystalline form that was non-hygroscopic below 70% RH (0.5% gain). TGA showed about 3% weight loss from the sample below about 100° C.

The hydrochloride salt was polymorphic, adopting crystalline forms exhibiting DSC endotherms at 119° C. (Form III), 127° C. (Form IV), 167° C. (Form II), and 186° C. (Form I). Another form, potentially an ethanol solvate/hydrate, exhibited multiple endotherms, corresponding to 1) desolvation at about 100° C., 2) Form I at about 183° C., and 3) possibly another polymorph at about 200° C. Table 2 below illustrates certain characteristics of observed hydrochloride salt crystal forms:

TABLE 2

| | Crystal Form | Crystal Form | Crystal | |
|---|---|---|---|---|
| Crystal Form I | II | III | Form IV | Crystal Form V |
| Mono- | | | | |
| hydrochloride | | | | |
| (8% HCl) | | | | |
| Melting: | Melting: | Melting: | Melting: | Three peaks: |
| 180-186° C. | 165° C. | 125° C. | 125° C. | About 100° C. |
| | | | | About 180° C. |
| | | | | About 200° C. |
| Non- | Somewhat | Hygroscopic | Not tested | Hygroscopic |
| hygroscopic | hygroscopic | (10% water | | (7% at RH |
| (see FIG. 9) | (5% water at | at RH 50%; | | 50%; see |
| | RH 50%; see | see FIG. 11) | | FIG. 12) |
| | FIG. 10) | | | |

Of the various observed hydrochloride forms, only Form I (186° C.) is relatively non-hygroscopic, gaining only about 0.5% moisture when equilibrated at RH less than or equal to 70%. At 70-100% RH, Form I gains at least about 2% moisture, but loses it without significant hysteresis on decreasing RH. Evidence of a hydrochloride hydrate was not observed after the hygroscopicity test.

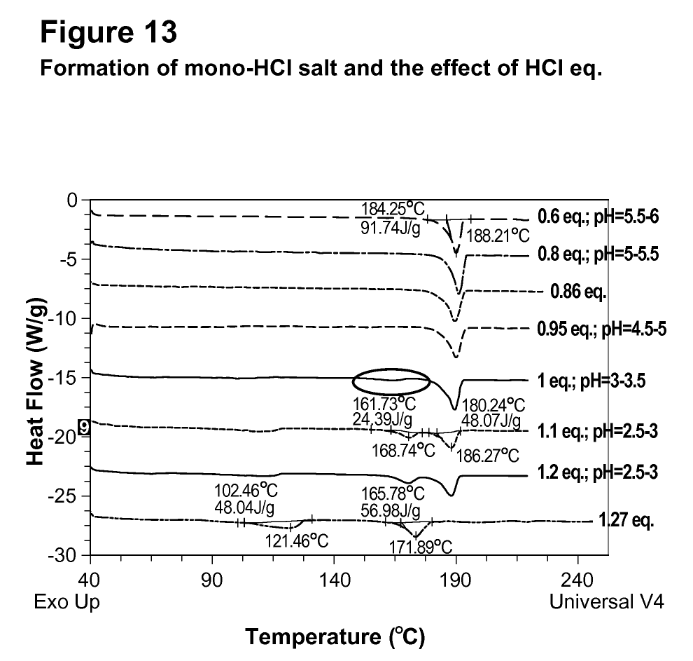

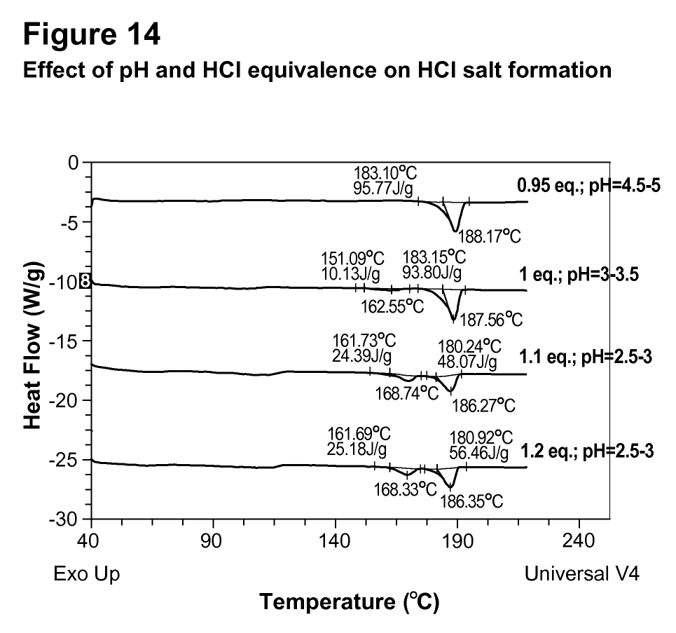

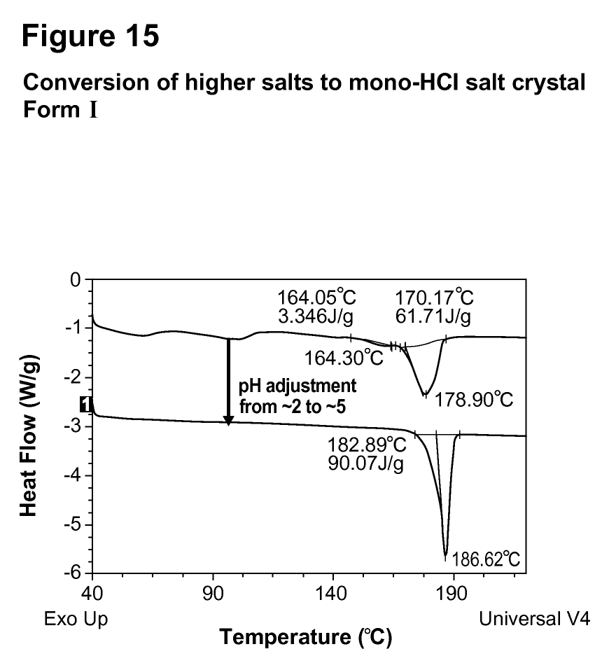

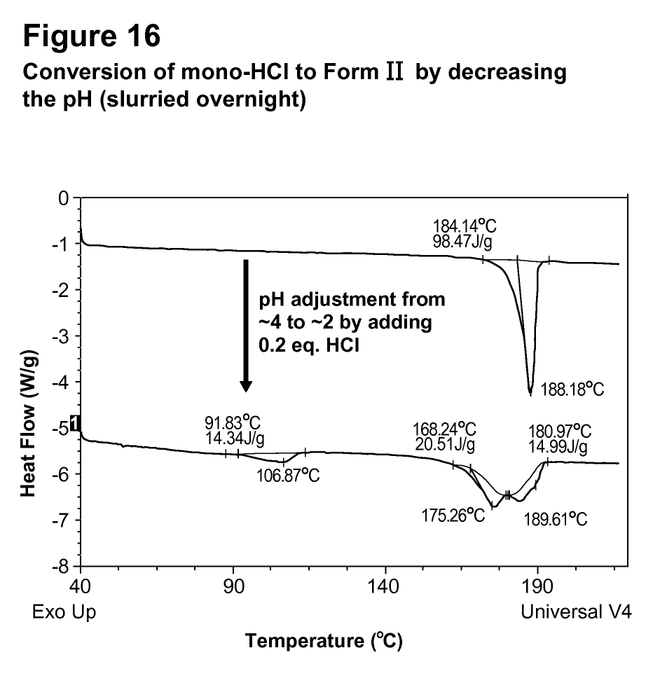

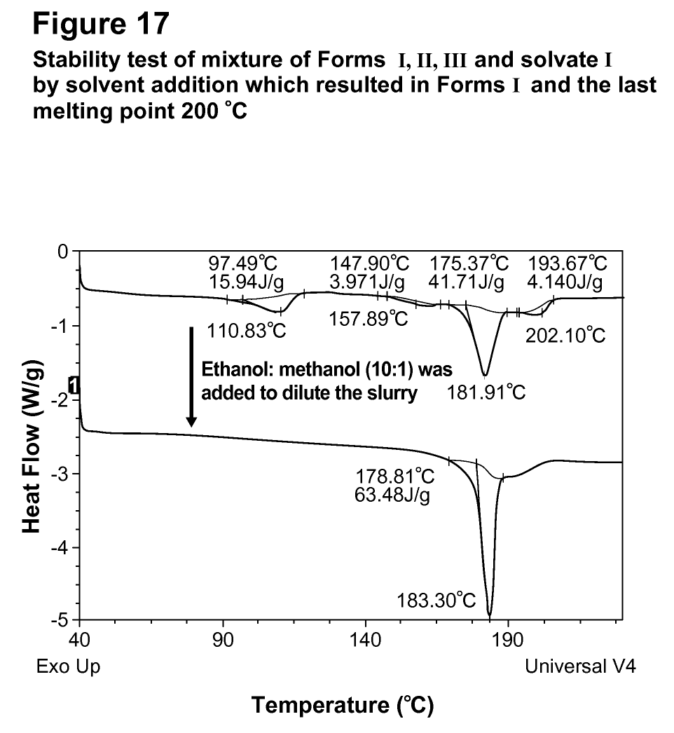

Higher degrees of hydrochloride salt were formed, depending on the amount of hydrochloric acid present in the solution during reactive crystallization. The conversion of higher degrees of hydrochloride salt to mono-hydrochloride salt can be achieved by adjusting the pH of the solution to more than pH 5. Further adjustment, however, can result in formation of inorganic salts. In some embodiments, pure mono-hydrochloride salt forms are produced with hydrochloride equivalence and slurry pH of <0.95 eq. (e.g., 0.93) and pH 5, respectively (see, for example, FIGS. 13-16).

Both Form III and Form I are soluble in MC/T to >100 mg/ml and therefore are fully soluble. Thus, would not expect pharmacokinetic variations upon administration of these forms.

### Example 4

### Characterization of Certain Crystal Forms of Hydrochloride Salt

The present Example describes characterization of two surprisingly non-hygroscopic crystal forms (Forms I and II, as described above in Example 2) of a hydrochloride salt of a compound of formula 1. Both forms are considerably soluble in water. The melting point of Form I is 185° C. (plus or minus 5 degrees); the melting point of Form II is 166° C. (plus or minus 2 degrees).

Form I picks up moisture at relative humidity (RH) of about 50% and absorbs up to about 2% water eventually (90% RH) and loses the water as RH decreases (<50%). Form I also exhibits characteristic X-ray peaks at 2θ of 15.3° and 21.9°, plus or minus about 0.3°, depending upon the machine and measurement method utilized.

Form II picks up moisture at RH of about 20% and absorbs up to 7% water eventually (RH of 90%) and holds 2% at low RH (0%). Form II also exhibits characteristic X-ray peaks at 20 of 20.2° and 24.9°, plus or minus about 0.3°, depending upon the machine and measurement method utilized. Differential scanning calorimetry data were collected for each solid form achieved using a DSC (TA instruments, model Q1000) under the following parameters: 50 mL/min purge gas (N₂); scan range 40 to 200° C., scan rate 10° C./min.

Thermogravimetric analysis data were collected using a TGA instruments (Mettler Toledo, model TGA/SDTA 851e) under the following parameters: 40 ml/min purge gas (N₂); scan range 30 to 250° C., scan rate 10° C./min.

X-ray data were acquired using an X-ray powder diffractometer (Bruker-axs, model D8 advance) having the following parameters: voltage 40 kV, current 40.0 mA, scan range (2θ) 3.7 to 30°, scan step size 0.0°, total scan time 33 minutes, VANTEC detector, and antiscattering slit 1 mm.

Dynamic Vapor Sorption (DVS) was done at 25-26° C.

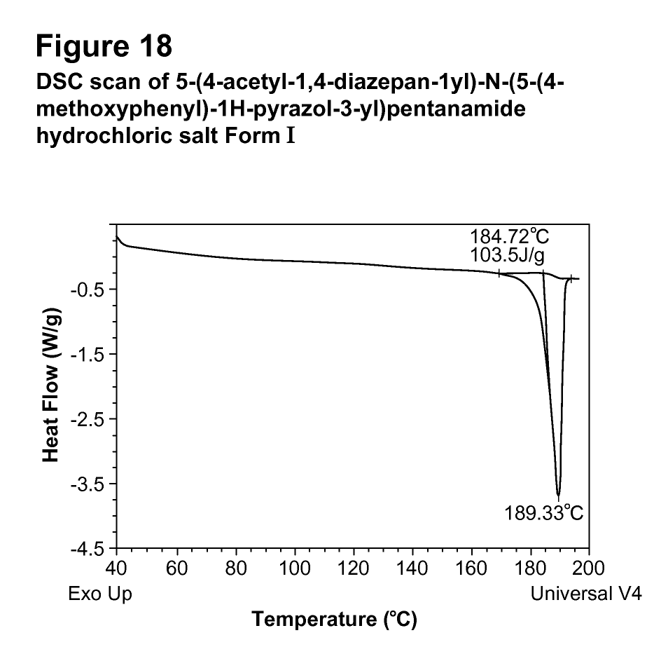

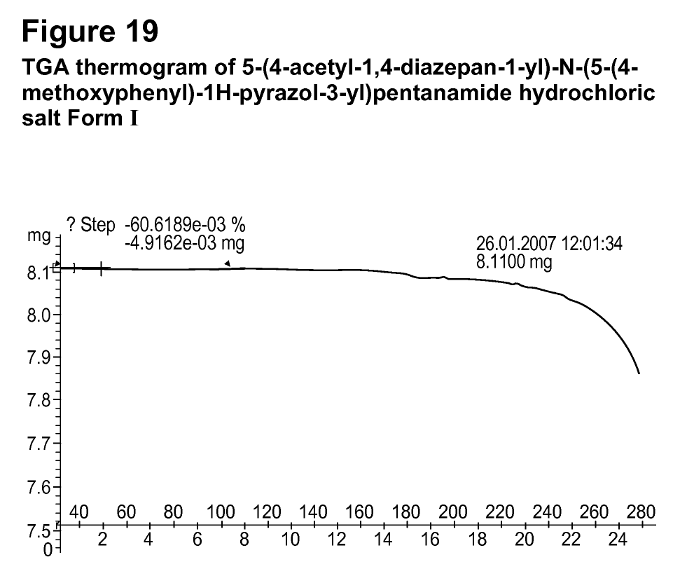

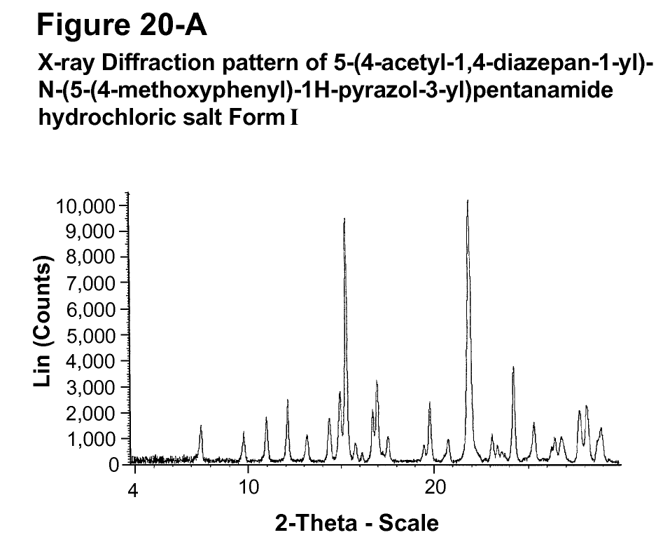

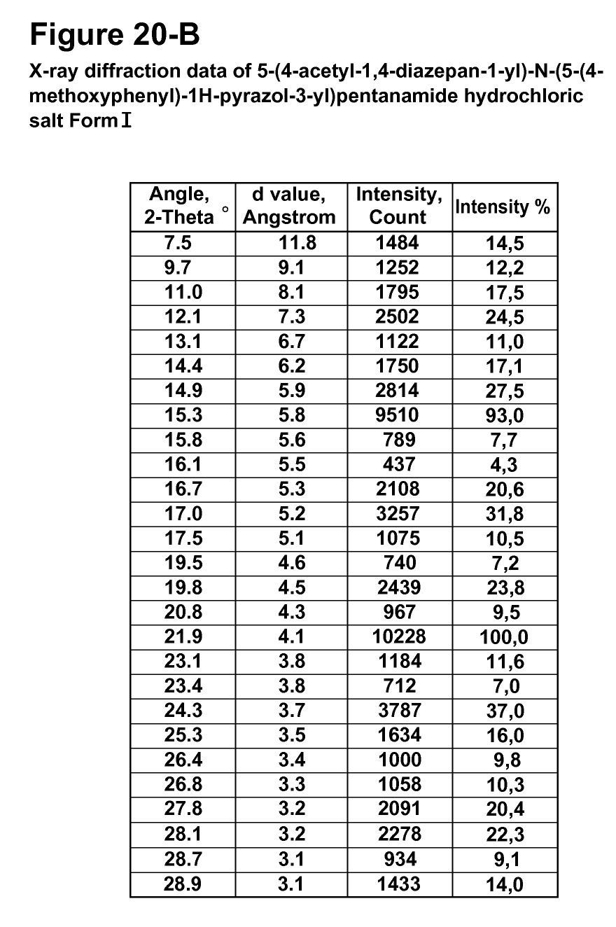

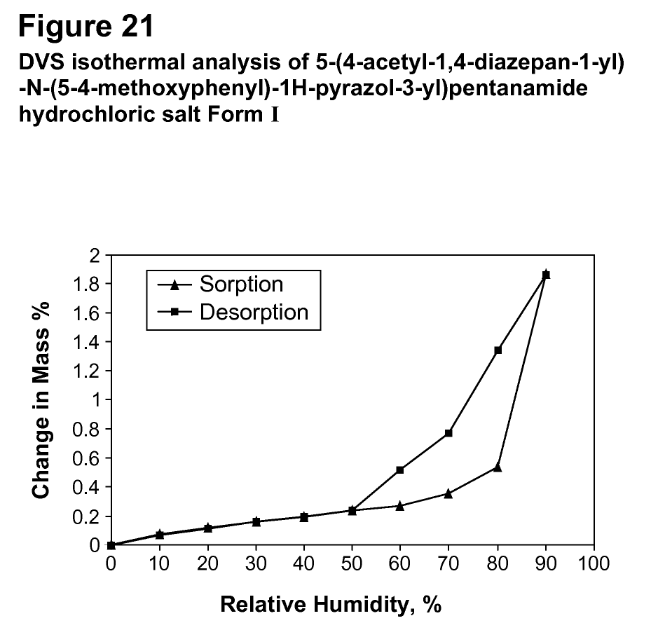

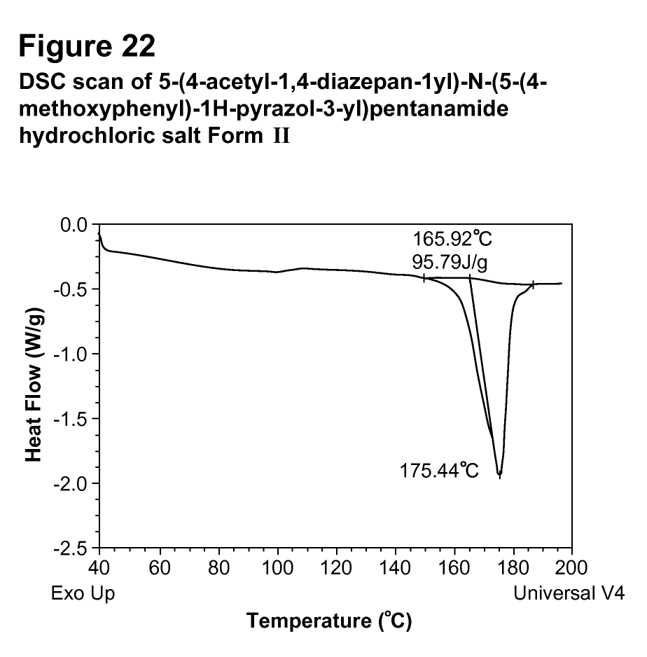

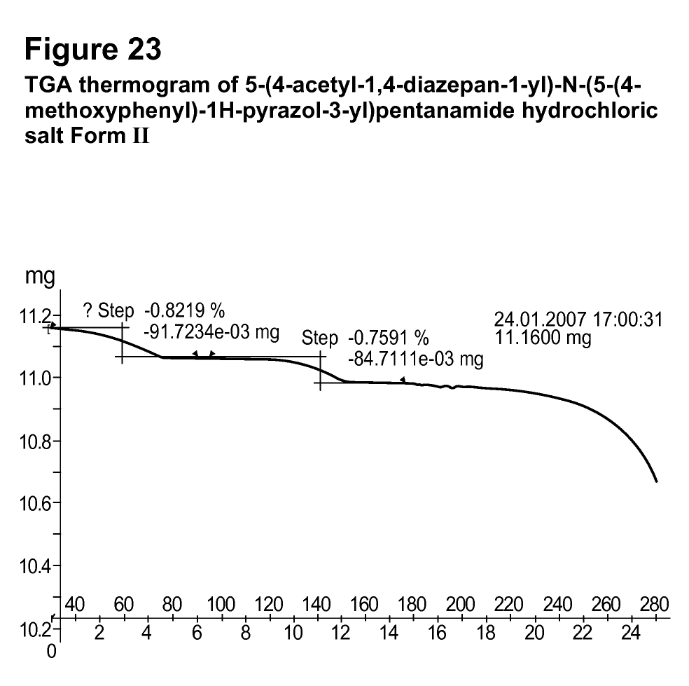

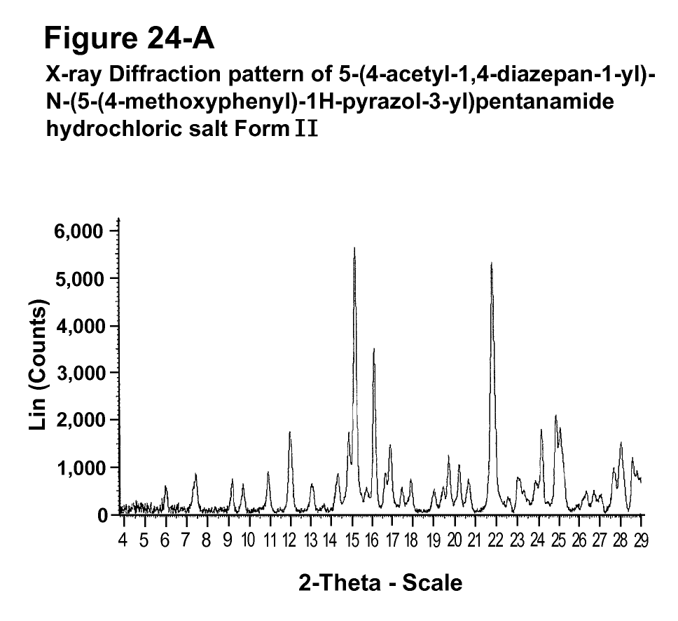

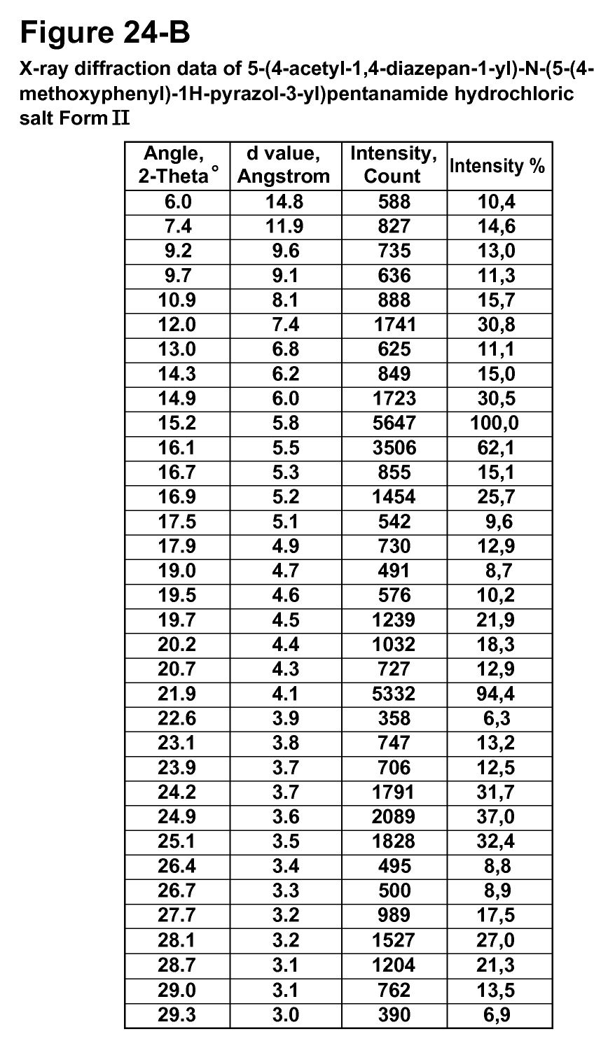

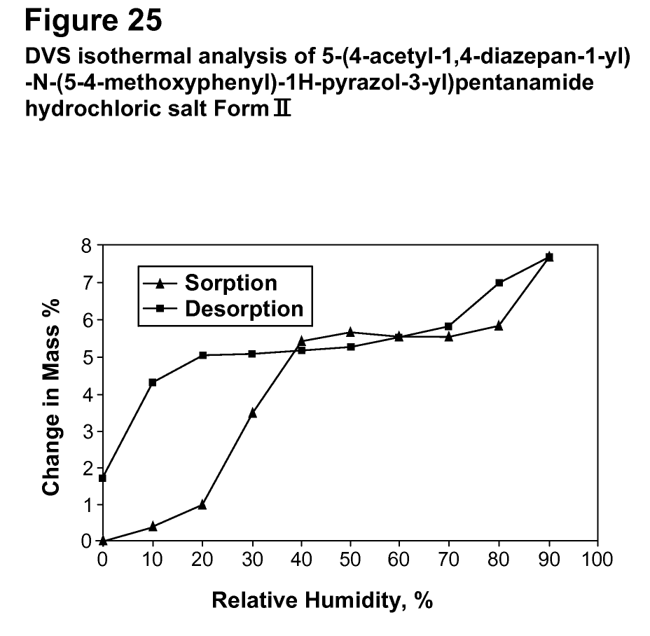

Results of thermal studies on Crystal Forms I and II of the hydrochloride salt of a compound of formula 1 are included in FIGS. 18-25.

### Example 5

### X-Ray Characterization of Crystal Form I of the Hydrochloride Salt of a Compound of Formula 1

The present Example presents the results of X-ray characterization of crystal Form I of the hydrochloride salt of a compound of formula 1.

### Data Collection

A colorless plate crystal of C₂₂H₃₂ClN₅O₃ having approximate dimensions of 0.55×0.18×0.06 mm was mounted on a loop. All measurements were made on a Rigaku RAXIS SPIDER imaging plate area detector with graphite monochromated Cu—Kα radiation. Long exposures and careful integration of the images were utilized to obtain the data set.

Indexing was performed from 4 oscillations that were exposed for 60 seconds. The crystal-to-detector distance was 127.40 mm.

Cell constants and an orientation matrix for data collection corresponded to a primitive triclinic cell with dimensions:

a=7.6642(7)Å α=79.933(5)°

b=12.2271(14)Å β=75.917(4)°

c=12.6614(11)Å γ=78.050(5)°

V=1116.19(19)Å³

For Z=2 and F.W.=449.98, the calculated density is 1.339 g/cm³. Based on a statistical analysis of intensity distribution, and the successful solution and refinement of the structure, the space group was determined to be:

P−1(#2)

The data were collected at a temperature of −173±1° C. to a maximum 20 value of 136.5°. A total of 148 oscillation images were collected. A sweep of data was done using \acute{ω} scans from 20.0 to 200.0° in 5.0° steps, at χ=0.0° and Φ=0.0°. Additional sweeps were performed from 30.0 to 120.0° at χ=540.0° and Φ=0.0°, from 20.0 to 200.0° at χ=540.0° and Φ=90.0°, from 20.0 to 200.0° at χ=540.0° and Φ=180.0°, and from 20.0 to 130.0° at χ=540.0° and Φ=270.0°. The exposure rate was 48.0 [sec./°]. The crystal-to-detector distance was 127.40 mm. Readout was performed in the 0.100 mm pixel mode.

### Data Reduction

Of the 10254 reflections that were collected, 3669 were unique (Rᵢₙₜ=0.220).

The linear absorption coefficient, μ, for Cu—Kα radiation is 17.956 cm⁻¹. The data were corrected for Lorentz and polarization effects.

### Structure Solution and Refinement

The structure was solved by direct methods (Altomare, et al. J. Appl. Cryst., 27, 435, 1994) and expanded using Fourier techniques (Beurskens, et al. The DIRDIF-99 program system, Technical Report of the Crystallography Laboratory, University of Nijmegen, The Netherlands. 1999). The non-hydrogen atoms were refined anisotropically. Hydrogen atoms were included but not refined. The final cycle of full-matrix least-squares refinement¹ on F² was based on 3669 observed reflections and 282 variable parameters and converged (largest parameter shift was 0.00 times its esd) with unweighted and weighted agreement factors of: ¹ Least Squares function minimized: (SHELXL97) ΣW(F_{O}²−F_{c}²)² where w=Least Squares weights.² Standard deviation of an observation of unit weight: [EW(F_{O}²−F_{c}²)²/(N_{O}−N_{V}]^{1/2}, where: N_{O}=number of observations; N_{V}=number of variables

R1=Σ∥Fo|−|Fc∥/Σ|Fo|=0.1168

wR2=[Σ(w(Fo²−Fc²)²)/w(Fo²)²]½=0.2323

The standard deviation of an observation of unit weight² was 0.96. Unit weights were used. The maximum and minimum peaks on the final difference Fourier map corresponded to 0.50 and −0.26 e−/Å³, respectively.

Neutral atom scattering factors were taken from Cromer and Waber (Cromer & Waber, “International Tables for X-ray Crystallography”, Vol. IV, The Kynoch Press, Birmingham, England, Table 2.2 A, 1974). Anomalous dispersion effects were included in Fcalc (Ibers & Hamilton, Acta Crystallogr., 17, 781, 1964); the values for Δf and Δf″ were those of Creagh and McAuley (Creagh & McAuley, “International Tables for Crystallography”, Vol C, (A. J. C. Wilson, ed.), Kluwer Academic Publishers, Boston, Table 4.2.6.8, pages 219-222, 1992). The values for the mass attenuation coefficients are those of Creagh and Hubbell (Creagh & Hubbell, “International Tables for Crystallography”, Vol C, (Wilson, ed.), Kluwer Academic Publishers, Boston, Table 4.2.4.3, pages 200-206, 1992). All calculations were performed using the Crystal Structure crystallographic software package (Crystal Structure Analysis Package, Rigaku and Rigaku/MSC (2000-2006); 9009 New Trails Dr. The Woodlands TX 77381 USA) except for refinement, which was performed using SHELXL-97 (Sheldrick, G. M., 1997).

### Experimental Details

A. Crystal Data

| | |
|---|---|
| Empirical Formula | C₂₂H₃₂CIN₅O₃ |
| Formula Weight | 449.98 |
| Crystal Color, Habit | colorless, plate |
| Crystal Dimensions | 0.55 × 0.18 × 0.06 mm |
| Crystal System | Triclinic |
| Lattice Type | Primitive |
| Indexing Images | 4 oscillations @ 60.0 seconds |
| Detector Position | 127.40 mm |
| Pixel Size | 0.100,, |
| Lattice Parameters | a = 7.6642(7) Å |
| | b = 12.2271(14) Å |
| | c = 12.6614(11) Å |
| | α = 79.933(5) ° |
| | β = 75.917(4) ° |
| | γ = 78.050(5) ° |
| | V = 116.19(19) Å³ |
| Space Group | P-1 (#2) |
| Z value | 2 |
| D_{calc} | 1.339 g/cm³ |
| F₀₀₀ | 480.00 |
| μ(CuKα) | 17.956 cm⁻¹ |

B. Intensity Measurements

| | |
|---|---|
| Diffractometer | Rigaku RAXIS-SPIDER |
| Radiation | CuKα (λ = 1.54187 Å) |
| | graphite monochromated |
| Detector Aperture | 280 mm × 256 mm |
| Data Images | 148 exposures |
| ω oscillation Range (χ = 0.0, Φ = 0.0) | 20.0-200.0° |
| ω oscillation Range (χ = 54.0, Φ = 0.0) | 30.0-120.0° |
| ω oscillation Range (χ = 54.0, Φ = 90.0) | 20.0-200.0° |
| ω oscillation Range (χ = 54.0, Φ = 180.0) | 20.0-200.0° |
| ω oscillation Range (χ = 54.0, Φ = 270.0) | 20.0-130.0° |
| Exposure Rate | 48.0 sec./° |
| Detector Position | 127.40 mm |
| Pixel Size | 0.100 mm |
| 2θₘₐₓ | 136.5° |
| No. of Reflections Measured | Total: 10254 |
| | Unique: 3669 (Rᵢₙₜ = 0.220) |
| Corrections | Lorentz-polarization |
| | Absorption |

C. Structure Solution and Refinement

| | |
|---|---|
| Structure Solution | Direct Methods (SIR92) |
| Refinement | Full-matrix least-squares |
| | on F² |
| Function Minimized | Σw(Fo² − Fc²)² |
| Lease Squares Weights | w = |
| | 1/[σ²(Fo²) + (0.0050 . P)²] |
| | Where |
| | P = (Max(Fo², 0) + 2Fc²)/3 |
| 2θₘₐₓ cutoff | 136.5° |
| Anomalous Dispersion | All non-hydrogen atoms |
| No. Observations (All reflections) | 3669 |
| No. Variables | 282 |
| Reflection/Parameter Ratio | 13.01 |
| Residuals: R1 (I > 2.00σ(I)) | 0.1168 |
| Residuals: R (All reflections) | 0.1865 |
| Residuals wR2 (All reflections) | 0.2323 |
| Goodness of Fit Indicator | 0.959 |
| Max Shift/Error in Final Cycle | 0.002 |
| Maximum peak in Final Diff. Map | 0.50 e−/Å³ |
| Minimum peak in Final Diff. Map | −0.26 e−/Å³ |

### Example 6

### Preparation of Crystal Form I of the Hydrochloride Salt of a Compound of Formula 1

The present Example describes the preparation of crystal form I of the hydrochloride salt of a compound of formula 1.

611.7 mg of the free base form of a compound of formula 1 was dissolved in 1.97 mL acetone at 35° C. A solution of 5% HCl in acetone-water was prepared by diluting 37.5% aq. HCL using acetone. 0.6 ml of 5% HCl was added slowly. 1.2 ml EtOH ASDQ (100:10 ethanol:methanol) was added slowly. The solution became milky in a few minutes; stirring was performed for around 5 minutes. 0.25 ml of 5% HCl was added slowly. After 5 minutes, 0.25 ml of 5% HCl was added slowly. After 5 minutes, 0.087 ml of 5% HCl was added slowly. The mixture was heated to about 40-50° C. The mixture was left at room temperature while stirring overnight. Crystals were filtered and washed with 2 ml acetone, and were dried at 45° C. for about 7 hours. 505 mg of solid were recovered.

### Example 7

### Preparation of Crystal Form I of the Hydrochloride Salt of a Compound of Formula 1

The present Example describes the preparation of crystal form I of the hydrochloride salt of a compound of formula 1.

377 mg of the free base form of a compound of formula 1 was dissolved in 1.2 ml acetone at 35° C. 0.754 ml ethanol ASDQ (100:10 ethanol:methanol) was added. A solution of 5% HCl in acetone-water was prepared by diluting 37.5% aq HCl using acetone. 0.18 ml diluted HCl solution was added slowly. A seed of crystal form I of a the hydrochloride salt of a compound of formula 1 was added. 0.18 ml diluted HCl solution was added slowly. Around two minutes later, 0.18 ml diluted HCl solution was added slowly. Around two minutes later, another 0.18 ml diluted HCl solution was added slowly. The mixture was heated to about 40-50° C., and then was left at room temperature while stirring overnight. The crystals were filtered and washed with 1.5 ml acetone, and were dried at 45° C. for about 6 hours.

### Example 8

### Preparation of Crystal Form II of the Hydrochloride Salt of a Compound of Formula 1

The present Example describes the preparation of crystal form II of the hydrochloride salt of a compound of formula 1.

108.6 mg of free base of a compound of formula 1 was dissolved in 0.85 ml acetone:water (99:1 vol) at 35° C. 1.2 eq HCl (228 mg 5 wt % HCl in water-acetone) was added in 5 minutes [5% acid was made using 37.5% aq acid and diluting with acetone:water (99:1)]. After a few minutes, it oiled out and a sticky gel formed. The mixture was heated to 50° C.; gel was still present. 0.25 ml ethanol ASDQ was added. In about 15 minuted the slurry started getting white. The heater was turned off and the mixture was stirred overnight at room temperature. Crystals were filtered and washed with acetone, and were dried at 40-45° C. under full vacuum for a few hours.

### Example 9

### Alternative Purification of the Hydrochloride salt of 5-(4-Acetyl-[1,4]diazepan-1-yl)-pentanoic acid [5-(4-methoxy-phenyl)-2H-pyrazol-3-yl]-amide

The hydrochloride salt of 5-(4-Acetyl-[1,4]diazepan-1-yl)-pentanoic acid [5-(4-methoxy-phenyl)-2H-pyrazol-3-yl]-amide may be re-purified by basifying the hydrochloride salt and extracting the free base into a suitable solvent (eg. methylene chloride). The organic extracts may be washed with water. The organic phase is concentrated and the solvent switched to ethanol. Acetone is added to give a solution of the free base which was clarified and mixed with ethanol, acetone, hydrochloric acid, and water. Acetone is added, the solids filtered, washed with mixture of acetone, water and dried to give the title compound. A representative procedure is described below.

To a suitable reactor, the hydrochloride salt of 5-(4-Acetyl-[1,4]diazepan-1-yl)-pentanoic acid [5-(4-methoxy-phenyl)-2H-pyrazol-3-yl]-amide (0.2 kg) was dissolved in water (0.80 L) and clarified through a filter pad. To the filtrates was added methylene chloride (2.65 kg) and cooled to 15° C. A 30% aqueous solution of sodium hydroxide (0.062 kg) was added over 30 mins and mixed for 20 mins. The pH was >8. The layers were separated; the organic layer was washed with water (2×0.40 kg) and distilled down to 0.46 L forming a hazy mixture. The methylene chloride solvent was exchanged with ethanol by vacuum distillation chases (2×0.79 kg).

Acetone (0.63 kg) was added to the concentrate and the solution clarified. An accurate strength of the free base was determined of the concentrate. Water (0.065 kg) was added to form a clear solution. A solution of 5% HCl (0.043 kg) in acetone (0.14 kg) and alcohol (0.14 kg of ethanol:acetone (91:9) v/v) was prepared and stirred until homogeneous at 10° C. About one third of the 5% HCl solution (0.098 kg) was added to the reactor over a minimum of 20 min., maintaining the temperature in the range of 20-25° C. A second third of the 5% HCl solution (0.098 kg) was then added to the reactor over a minimum of 20 min., maintaining the temperature in the range of 20-25° C. The contents of the reactor were seeded with 75 mg of 5-(4-acetyl-[1,4]diazepan-1-yl)-N-[5-(4-methoxy-phenyl)-1H-pyrazol-3-yl]-pentanamide HCl (e.g., Form 1), followed by the addition of the last third of 5% HCl solution (0.098 kg) over a minimum of 20 min., maintaining the temperature in the range of 20-25° C. The contents of the reactor were seeded with another 75 mg of 5-(4-acetyl-[1,4]diazepan-1-yl)-N-[5-(4-methoxy-phenyl)-1H-pyrazol-3-yl]-pentanamide HCl (e.g., Form 1). Another 0.08 equiv. of the 5% HCl solution (0.029 kg) was then added to the reactor over a minimum of 30 min., maintaining the temperature in the range of 20-25° C. Judicious monitoring of pH was performed to attain the desired pH range of 5.2-5.8. The mixture was stirred at 20-25° C. for a minimum of 1 hr., forming a thin suspension. Acetone (0.63 kg) was added over a minimum of 60 min., maintaining the temperature in the range of 20-25° C. The mixture was stirred at 20-25° C. for a minimum of 60 min. Acetone (1.58 kg) was added to the reactor over a minimum of 3 hr., maintaining the temperature in the range of 20-25° C., forming a thick suspension. The mixture was then stirred at 20-25° C. for a minimum of 12 h. Crystallization was considered complete when there was ≦15% of the product present in the mother liquor. Longer stirring was employed if crystallization was not complete. The mixture was then filtered on a Buchner funnel (polypropylene pad) using house vacuum. A solution of water (0.012 kg), acetone (0.24 kg) and 0.063 kg alcohol (ethanol:acetone (91:9) v/v) was stirred until homogeneous (20% ethanol, 3% water, 77% acetone overall). This solution was used to wash the filter cake. A solution of water (0.012 kg), acetone (0.18 kg) and 0.13 kg alcohol (ethanol:acetone (91:9) v/v) was stirred until homogeneous (40% ethanol, 3% water, 57% acetone overall). This solution was used to wash the filter cake. The wet cake was subjected to suction under nitrogen using house vacuum and held for 30 min. after dripping stopped. Product purity was checked by HPLC and additional washing was performed if total impurities were not ≦2%. Product was oven dried in a vacuum oven with nitrogen bleed at 38-45° C., maintaining vacuum at 20 torr for a minimum of 12 h until loss on drying of less than 1% was obtained. Following drying, 0.17 kg of the title compound was obtained in 85% yield.

### Example 10

### Biological Activity

### Cloning of α7 Nicotinic Acetylcholine Receptor and Generation of Stable Recombinant α7 nAChR Expressing Cell Lines

Full length cDNAs encoding the α7 nicotinic acetylcholine receptor were cloned from a rat brain cDNA library using standard molecular biology techniques. Rat GH4C1 cells were then transfected with the rat receptor, cloned and analyzed for functional alpha7 nicotinic receptor expression employing a FLIPR assay to measure changes in intracellular calcium concentrations. Cell clones showing the highest calcium-mediated fluorescence signals upon agonist (nicotine) application were further subcloned and subsequently stained with Texas red-labelled α-bungarotoxin (BgTX) to analyse the level and homogeneity of alpha7 nicotinic acetylcholine receptor expression using confocal microscopy. Three cell lines were then expanded and one characterised pharmacologically (see Table 3 below) prior to its subsequent use for compound screening.

TABLE 3
Pharmacological characterization of α7 nAChR stably
expressed in GH4C1 cells using the functional FLIPR assay

| Compound | EC₅₀ [microM] |
|---|---|
| Acetylcholine | 3.05 ± 0.08 (n = 4) |
| Choline | 24.22 ± 8.30 (n = 2) |
| Cytisine | 1.21 ± 0.13 (n = 5) |
| DMPP | 0.98 ± 0.47 (n = 6) |
| Epibatidine | 0.012 ± 0.002 (n = 7) |
| Nicotine | 1.03 ± 0.26 (n = 22) |

### Development of a Functional FLIPR Assay for Primary Screening

A robust functional FLIPR assay (Z′=0.68) employing the stable recombinant GH4C1 cell line was developed to screen the α7 nicotinic acetylcholine receptor. The FLIPR system allows the measurements of real time Ca²⁺-concentration changes in living cells using a Ca²⁺ sensitive fluorescence dye (such as Fluo4). This instrument enables the screening for agonists and antagonists for alpha 7 nAChR channels stably expressed in GH4C1 cells.

### Cell Culture

GH4C1 cells stably transfected with rat-α7-nAChR (see above) were used. These cells are poorly adherent and therefore pretreatment of flasks and plates with poly-D-lysine was carried out. Cells are grown in 150 cm² T-flasks, filled with 30 ml of medium at 37° C. and 5% CO₂.

### Data Analysis

EC₅₀ and IC₅₀ values were calculated using the IDBS XLfit4.1 software package employing a sigmoidal concentration-response (variable slope) equation:

Y=Bottom+((Top−Bottom)/(1+((EC₅₀/X)̂HillSlope))

### Assay Validation

The functional FLIPR assay was validated with the α7 nAChR agonists nicotine, cytisine, DMPP, epibatidine, choline and acetylcholine. Concentration-response curves were obtained in the concentration range from 0.001 to 30 microM. The resulting EC₅₀ values showed a rank order of agonists is in agreement with published data (Quik et al., 1997, Mol. Pharmacol., 51, 499-506).

The assay was further validated with the specific α7 nAChR antagonist MLA (methyllycaconitine), which was used in the concentration range between 1 microM to 0.01 nM, together with a competing nicotine concentration of 10 microM. The IC₅₀ value was calculated as 1.31±0.43 nM in nine independent experiments.

### Development of Functional FLIPR Assays for Selectivity Testing

Functional FLIPR assays were developed in order to test the selectivity of compounds against the α1 (muscular) and α3 (ganglionic) nACh receptors and the structurally related 5-HT3 receptor. For determination of activity at α1 receptors natively expressed in the rhabdomyosarcoma derived TE 671 cell line an assay employing membrane potential sensitive dyes was used, whereas α3 selectivity was determined by a calcium-monitoring assays using the native SH-SY5Y cell line. In order to test selectivity against the 5-HT3 receptor, a recombinant cell line was constructed expressing the human 5-HT3A receptor in HEK 293 cells and a calcium-monitoring FLIPR assay was employed.

### Screening of Compounds

Compounds of formula 1, in appropriate forms, can be tested using the functional FLIPR primary screening assay employing the stable recombinant GH4C1 cell line expressing the α7 nAChR. Potency and selectivity (e.g., against the α1 nAChR, α3 nAChR and 5HT3 receptors) can be demonstrated. In some embodiments, an EC50 within the range of about 1-2 nM is observed.

### EQUIVALENTS

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any targeting moiety, any disease, disorder, and/or condition, any linking agent, any method of administration, any therapeutic application, etc.) can be excluded from any one or more claims, for any reason, whether or not related to the existence of prior art.

Publications discussed above and throughout the text are provided solely for their disclosure prior to the filing date of the present application. Nothing herein is to be construed as an admission that the inventors are not entitled to antedate such disclosure by virtue of prior disclosure.

1. A compound having the structure shown below: 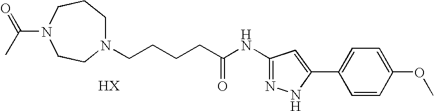
wherein the salt is fumarate.

2. A compound having the structure shown below: 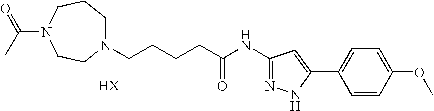
wherein the salt is sulfate.

3. A compound having the structure shown below: 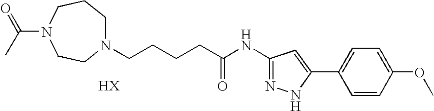
wherein the salt is D-glucoronate.

4. A compound having the structure shown below: 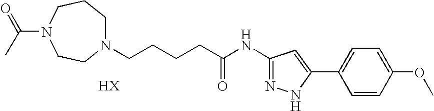
wherein the salt is hydrochloride.

5. The compound of claim 4, in crystalline form.

6. The compound of claim 4, in crystalline form characterized by one or more X-ray diffraction peaks as shown in FIG. 6 that are characteristic of Form I.

7. The compound of claim 4, in crystalline form characterized by one or more X-ray diffraction peaks as shown in FIG. 6 that are characteristic of Form II.

8. The compound of claim 4, in crystalline form characterized by one or more X-ray diffraction peaks as shown in FIG. 6 that are characteristic of Form III.

9. The compound of claim 4, in crystalline form characterized by one or more X-ray diffraction peaks as shown in FIG. 6 that are characteristic of Form IV.

10. The compound of claim 4, in crystalline form characterized by one or more X-ray diffraction peaks as shown in FIG. 6 that are characteristic of Form V.

11. The compound of claim 6 wherein the characteristic peaks include one or more peaks at about 2θ of 15.3° and 21.9°.

12. The compound of claim 7 wherein the characteristic peaks include one or more peaks at about 2θ of 20.2° and 24.9°.

13. A composition comprising the compound of any one of claims 1-12.

14. The composition of claim 13, which is a solid composition.

15. The composition of claim 13, wherein the compound is present in solid form.

16. A composition comprising a salt form of a compound of formula 1, wherein substantially all of the compound in the composition is in a solid form.

17. A composition comprising a salt form of a compound of formula 1, wherein the compound in the composition is present in a form selected from the group consisting of fumarate, sulfate, D-glucoronate, hydrochloride and combinations thereof.

18. A composition comprising a compound of formula 1, wherein at least some of the compound present in the composition is present in a salt form selected from the group consisting of fumarate, sulfate, D-glucoronate, hydrochloride and combinations thereof.

19. A composition comprising a compound of formula 1, wherein at least some of the compound present in the composition is in a crystalline form.

20. A pharmaceutical composition comprising:
a therapeutically effective amount of a salt form of a compound of formula 1; and
at least one pharmaceutically acceptable carrier or excipient.

21. The pharmaceutical composition of claim 20, which composition is formulated for oral delivery.

22. A method comprising steps of:
administering to a subject suffering from or susceptible to one or more psychotic diseases, neurodegenerative diseases involving a dysfunction of the cholinergic system, or conditions of memory or cognition impairment a pharmaceutical composition comprising:
a therapeutically effective amount of a salt form of a compound of formula 1; and
at least one pharmaceutically acceptable carrier or excipient.

23. A method for improving or stabilizing cognitive function in a subject comprising administering to the subject a pharmaceutical composition comprising:
a therapeutically effective amount of a salt form of a compound of formula 1; and
at least one pharmaceutically acceptable carrier or excipient.

24. A method comprising steps of:
administering to a subject suffering from or susceptible to one or more central nervous system (CNS) diseases or disorders a pharmaceutical composition comprising:
a therapeutically effective amount of a salt form of a compound of formula 1; and
at least one pharmaceutically acceptable carrier or excipient.

25. The method of claim 24, wherein the disease or disorder is selected from the group consisting of psychoses, anxiety, senile dementia, depression, epilepsy, obsessive compulsive disorders, migraine, cognitive disorders, sleep disorders, feeding disorders, anorexia, bulimia, binge eating disorders, panic attacks, disorders resulting from withdrawal from drug abuse, schizophrenia, gastrointestinal disorders, irritable bowel syndrome, memory disorders, Alzheimer's disease, Parkinson's disease, Huntington's chorea, schizophrenia, attention deficit hyperactive disorder, neurodegenerative diseases characterized by impaired neuronal growth, and pain.

26. The method of claim 22, 23, or 24, wherein at least some of the administered compound is present in a crystalline form.

27. The composition of any one of claims 13 through 21, in unit dosage form.
